(19) United States
(12) Patent Application Publication
(10) Pub. No.: US 2026/0258379 A1
(43) Pub. Date: Sep. 3, 2026

(54) PHOTOSENSITIVE DNA POLYMERASE, USE METHOD AND USE THEREOF

(71) Applicant: HEBEI AGRICULTURAL UNIVERSITY, Baoding (CN)

(72) Inventors: Qi CHENG, Baoding (CN); Yang WU, Baoding (CN); Yukun TIAN, Baoding (CN); Xizhe SUN, Baoding (CN); Lifeng HOU, Baoding (CN); Fanli ZENG, Baoding (CN)

(21) Appl. No.: 19/299,705

(22) Filed: Aug. 14, 2025

(30) Foreign Application Priority Data
Feb. 20, 2025 (CN) 202510189106.9

(51) Int. Cl. C12N 9/12; C07K 1/00; C07K 19/00; C12N 9/00; C12N 15/62; C12Q 1/6806; C12Q 1/6844

(52) U.S. Cl. CPC C12N 9/1241; C07K 1/00; C07K 19/00; C12N 9/93; C12N 15/62; C12Q 1/6806; C12Q 1/6844; C12Y 601/01; C07K 2319/00

(57) ABSTRACT

A photosensitive DNA polymerase, use method and use thereof, relate to the technical field of enzyme engineering, and address the technical problems of providing a protein and converting same into the photosensitive DNA polymerase. The protein has the amino acid sequence set forth in SEQ ID NO: 3, where X is pAzF, an amino group of the pAzF is linked via a peptide bond to a carboxyl group of lysine at position 4 in SEQ ID NO: 3, and a carboxyl group of the pAzF is linked via a peptide bond to an amino group of histidine at position 5 in SEQ ID NO: 3.

### CROSS REFERENCE TO RELATED APPLICATION

This patent application claims the benefit and priority of Chinese Patent Application No. 202510189106.9 filed with the China National Intellectual Property Administration on Feb. 20, 2025, the disclosure of which is incorporated by reference herein in its entirety as part of the present application.

### REFERENCE TO SEQUENCE LISTING

A computer readable XML file entitled “GWP20240401000-Sequence Listing”, that was created on Jul. 23, 2025, with a file size of about 21,809 bytes, contains the sequence listing for this application, has been filed with this application, and is hereby incorporated by reference in its entirety.

### TECHNICAL FIELD

The present disclosure specifically relates to a photosensitive DNA polymerase, use method and use thereof.

### BACKGROUND

Currently, there are two main categories of nucleic acid amplification technologies. One category is polymerase chain reaction (PCR), which has been widely applied. Hot-start fluorescent quantitative PCR using a hot start DNA polymerase may be utilized for precise nucleic acid quantification and detection, and has been extensively applied in both laboratory and clinical settings. However, such one category of technology requires thermal cycling to drive PCR reactions, demanding more on equipment; and the other category of technology, isothermal nucleic acid amplification, may be conducted at room temperature with less demanding on equipment and faster reaction speeds. Nevertheless, existing methods for reaction initiation control primarily rely on the magnesium ion added, which poses challenges for precise control in large-scale reactions.

Similar issues exist in the real-time fluorescent polymerase chain reaction (RT-PCR) using conventional Taq DNA polymerase, which is widely used for nucleic acid detection and quantitative analysis. Taq DNA polymerase has a certain catalytic activity at room temperature, which can lead to non-specific amplification and subsequently cause deviations or even invalid test results. To address this issue, the mainstream solution at present is to use the monoclonal antibodies. The monoclonal antibodies bind to Taq DNA polymerase at low temperatures to inhibit the activity of the Taq DNA polymerase, while at high temperatures the monoclonal antibodies were inactive, and the activity of the Taq DNA polymerase was restored, thus realizing the hot start of fluorescence quantitative PCR and improving the accuracy of nucleic acid detection and quantitative analysis.

However, in the isothermal nucleic acid amplification technologies, all enzymes used are intolerant to high temperatures, making them unsuitable for hot-start modification. To achieve precise control of isothermal nucleic acid amplification, the existing technology often controls the reactions to start simultaneously by controlling the addition of magnesium ions, which serve as a crucial ion for isothermal nucleic acid amplification reactions. When there are multiple reactions, this technical approach realizes start control of the isothermal nucleic acid amplifications by preparing isothermal nucleic acid amplification systems without magnesium ions in advance, and adding the magnesium ions when the reaction initiation is required. However, the addition of the magnesium ions at last still need to be performed one by one; and if there are multiple reactions, there remains a time delay between the initiation of the first and last reactions, so the initiation still cannot be controlled very accurately.

However, as isothermal nucleic acid amplification rapidly advances, nucleic acid amplification at room temperature is possible with simple instrumentation and has broader application prospects. Among these technologies, recombinase aided isothermal nucleic acid amplification (also known as RAA) is one that perform nucleic acid amplification under isothermal conditions (37° C. to 42° C.) utilizing recombinase, single-stranded binding proteins, and DNA polymerase. The specific workflow involves the following steps: the recombinase, single-stranded DNA binding protein (SSB), and primers form a complex to search for the double-stranded DNA, deconjugate the double-stranded DNA at the sequence homologous to the primers. The single-stranded DNA binding protein prevents renaturation of the single-stranded DNA, while performing strand extension in the presence of DNA polymerase, ATP and dNTPs, thereby achieving nucleic acid amplification. Nevertheless, since isothermal nucleic acid amplification reactions may occur at room temperature, rendering the reactions occur one by one during the preparation of the isothermal nucleic acid amplification systems one by one. This poses critical challenges for controlling synchronized initiation of the multiple reactions. In application scenarios such as nucleic acid detection and quantitative analysis, where comparable results demand simultaneous initiation of all the amplification reactions. Therefore, the control of the initiation of all the nucleic acid amplification reactions is critical, which also cause limitation to the broader adoption of isothermal nucleic acid amplification technologies.

In summary, isothermal nucleic acid amplification technologies offer advantages such as more rapid reaction, simple temperature requirements, and flexible detection modalities. However, the lack of precise initiation control methods remains a critical bottleneck limiting its broader application. Consequently, it is a technical problem faced by those skilled in the art that providing a methodology to control over the initiation timing accurately of the isothermal nucleic acid amplification reaction to promote the application of the technologies in precise fields such as nucleic acid detection and quantitative analysis.

### SUMMARY

To address the aforementioned technical problems, the present disclosure modifies a DNA polymerase for isothermal nucleic acid amplification systems to confer photosensitivity. This modified photosensitive DNA polymerase is completely inactive without light illumination but becomes active only when it is irradiated by a specific wavelength of light. By employing such a photosensitive DNA polymerase, the isothermal nucleic acid amplification reaction systems remain inert during preparation. The reaction initiates exclusively after the preparation of all the systems followed by light irradiation of a specific wavelength, thereby achieving accurate light-initiated control over isothermal nucleic acid amplification. The light-initiated isothermal nucleic acid amplification technology based on photosensitive DNA polymerase enables synchronized initiation of multiple isothermal amplification systems, which eliminates experimental errors caused by asynchronous nucleic acid amplification initiation in precision-demanding experiments.

To solve the above technical problems, the present disclosure provides a protein, a salt thereof, or a derivative thereof, where
the protein is any one selected from the group consisting of:
B1) a protein having the amino acid sequence set forth in SEQ ID NO: 3;
B2) a protein having not less than 80% sequence identity to the protein of B1) and the same activity obtained by substitution, and/or deletion, and/or addition of an amino acid residue in the protein of B1); and
B3) a fusion protein obtained by linking a protein tag to an N-terminus and/or a C-terminus of the protein of B1) or the protein of B2); where
X is pAzF (azidophenylalanine), an amino group of the pAzF is linked via a peptide bond to a carboxyl group of the lysine at position 4 in SEQ ID NO: 3, and a carboxyl group of the pAzF is linked via a peptide bond to an amino group of the histidine at position 5 in SEQ ID NO: 3; and the pAzF has a structure shown as follows:

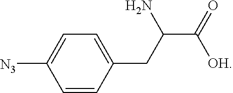 [SMILES: [N-]=[N+]=Nc1ccc(CC(N)C(=O)O)cc1]

In the above proteins, the term “protein-tag” refers to a polysaccharide hydrolase or protein co-expressed in fusion with the target protein through DNA in vitro recombination, which facilitates the expression, detection, tracing, and/or purification of the target protein. The protein tag may include a Flag tag, a His tag, an MBP tag, an HA tag, an myc tag, a GST tag, and/or a SUMO tag.

In the above proteins, “identity” refers to the identity between the amino acid sequences. Identity between the amino acid sequences may be determined using homology search sites on the international internet, such as the BLAST page on the NCBI homepage website. For example, a determination method includes: in advanced BLAST2.1, by using BlastP as the program, setting the Expect value to 10, setting all Filters to OFF, using BLOSUM62 as the Matrix, setting Gap existence cost, Per residue gap cost, and Lambda ratio to 11, 1, and 0.85 (default values), respectively, and conducting pairwise alignment of a pair of amino acid sequences to calculate the identity, thereby obtaining the percentage identity value (%).

In the above proteins, the “not less than 80% identity” may refer to at least 81%, 82%, 83%, 84%, 85%, 86%, 87%, 88%, 89%, 90%, 91%, 92%, 95%, 96%, 98%, 99%, or 100% identity.

In the above proteins, there are 602 amino acid residues in SEQ ID NO: 3.

In the present disclosure, one or more amino acids in the protein may be substituted with D-amino acids, artificially modified amino acids, and naturally occurring rare amino acids, to enhance the bioavailability, stability, and/or anticancer activity of the polysaccharide hydrolase. The D-amino acids refer to amino acids corresponding to L-amino acids that constitute proteins; the artificially-modified amino acids refer to common L-amino acids that constitute proteins modified by methylation, phosphorylation, or the like; and the naturally occurring rare amino acids include uncommon amino acids that constitute proteins and amino acids that do not constitute proteins, such as 5-hydroxylysine, methylhistidine, gamma-aminobutyric acid (GABA), and homoserine.

To solve the above technical problems, the present disclosure further provides a compound, a salt thereof, or a derivative thereof, where the compound has the following structural formula:

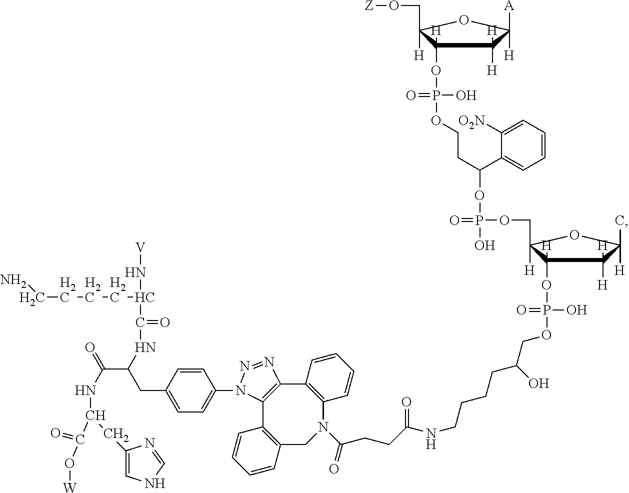

Where V is a polypeptide with an amino acid sequence of M(methionine) R (arginine); W is a polypeptide including residues 5 to 598 of SEQ ID NO: 3;

Z is an oligonucleotide strand including residues 1 to 19 of SEQ ID NO: 5; where C represents cytosine; A represents adenine;
a carboxyl group at a carboxyl terminus of the V forms a peptide bond with an amino group of a lysine residue; an amino group at an amino terminus of the W forms a peptide bond with a carboxyl group of the pAzF residue; and a phosphate group of a 3′-terminal cytosine deoxyribonucleotide of the Z forms a phosphodiester bond with a hydroxyl group on the C atom at position 5 of an adenine deoxyribonucleotide.

The present disclosure further provides a preparation method of the above proteins, including the step of transcribing and translating a nucleic acid having the nucleotide sequence set forth in SEQ ID NO: 4 to obtain the protein, where the reaction contains a pAzF-tRNA-TAG complex that specifically recognizes a TAG codon, and an amino arm of the pAzF-tRNA-TAG complex carries the pAzF.

Further, the preparation method includes the step of transcribing and translating the nucleic acid having the nucleotide sequence set forth in SEQ ID NO: 4 in vitro to obtain the protein, where the reaction contains an aminoacyl-tRNA synthetase capable of recognizing the pAzF and a tRNA configured to recognize a termination codon.

Further, the aminoacyl-tRNA synthetase capable of recognizing the pAzF and the tRNA configured to recognize the termination codon each are expressed via a pZA16-pAzF plasmid.

Further, the transcribing and translating are both conducted in a microorganism in any of the preparation methods above.

In the present disclosure, the microorganism may be Escherichia coli (E. coli). The E. coli may be an E. coli strain BL21 (DE3).

In the present disclosure, the pZA16-pAzF plasmid is purchased from AddGene (Catalog No. 31186).

In the present disclosure, the transcribing and translating in vitro of the nucleic acid with the nucleotide sequence set forth in SEQ ID NO: 4 is achieved through an expression vector. The expression vector may be a prokaryotic expression vector. The prokaryotic expression vector may be a pTrc99A vector. The pTrc99A vector is purchased from HonorGene (Catalog No. HG-VYA0262).

The present disclosure further provides a preparation method of the above compounds, including the step of obtaining the above compounds by subjecting an azide residue of the pAzF in the protein prepared by the preparation method to alkyne azide cycloaddition azide-alkyne cycloaddition reaction with an alkyne group of dibenzocyclooctyne in a photosensitive oligonucleotide strand; where the photosensitive oligonucleotide strand has the structural formula as follows:

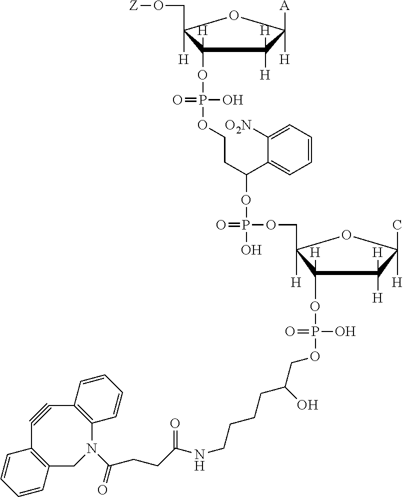

Where Z is the oligonucleotide strand including the residues 1 to 19 of SEQ ID NO: 5; C represents the cytosine; A represents the adenine;
the phosphate group of the 3′-terminal cytosine deoxyribonucleotide of the Z forms the phosphodiester bond with the hydroxyl group on the C atom at position 5 of the adenine deoxyribonucleotide.

To solve the above technical problems, the present disclosure further provides the following use:

The use is any one selected from the group consisting of:
1) use of the protein or the protein prepared by the preparation method described above in preparation of a photosensitive DNA polymerase; and
2) use of the protein or the protein prepared by the preparation method described above in isothermal nucleic acid amplification or in preparation of a product for the isothermal nucleic acid amplification.

To solve the above technical problems, the present disclosure further provides the following use:

The use is any one selected from the group consisting of:
1) use of the compound or the compound prepared by the preparation method described above in preparation of a photosensitive DNA polymerase product; and
2) use of the compound or the compound prepared by the preparation method described above in isothermal nucleic acid amplification or in preparation of a product for the isothermal nucleic acid amplification.

In the present disclosure, in addition to the photosensitive DNA polymerase, the photosensitive DNA polymerase product includes, but is not limited to, such as reaction buffer, dNTPs, Mg²⁺, stabilizers, enhancers, coenzymes or cofactors, dyes or tracers, and/or other additives.

In the present disclosure, the reaction buffer maintains the pH value and ionic strength of the reaction system to ensure the activity and stability of the DNA polymerase. The reaction buffer includes pH-stabilizing substances (e.g., Tris-HCl), ion strength regulators (e.g., KCl or (NH₄)₂SO₄) that influence activity of the DNA polymerase and primer binding, and/or detergents (e.g., Triton X-100 or NP-40) that is capable of preventing enzyme from binding to tube walls.

In the present disclosure, the magnesium ions are essential cofactor for DNA polymerase affecting primer binding, enzyme activity, and product specificity. In examples, magnesium ions may be provided as MgCl₂ or MgSO₄, either alone or in combination.

In the present disclosure, dNTPs (deoxyribonucleotide triphosphates, including dATP, dTTP, dCTP, and dGTP) serve as substrates for DNA synthesis). In examples, they may be pre-mixed in the reaction buffer or provided separately.

In the present disclosure, the stabilizers include but are not limited to glycerol and BSA (bovine serum albumin). Glycerol prevents enzyme inactivation during low-temperature storage. BSA (bovine serum albumin) may reduce tube wall adsorption and stabilize enzyme structure.

In the present disclosure, the enhancers include but are not limited to betaine and DMSO (dimethyl sulfoxide). The betaine may reduce DNA secondary structures (e.g., in high-GC regions). The DMSO (dimethyl sulfoxide) may promote DNA denaturation, which is suitable for the case of complex templates.

In the present disclosure, the coenzymes or cofactors include but are not limited to DTT (dithiothreitol) or β-mercaptoethanol, which may maintain a reducing environment to prevent enzyme oxidation and inactivation.

In the present disclosure, the dyes or tracers include but are not limited to bromophenol blue/xylene cyanol or ROX reference dye. Bromophenol blue/xylene cyanol acts as electrophoresis tracking dyes (which is pre-mixed in the buffer). ROX reference dye is used for fluorescence normalization in real-time quantitative PCR.

In the present disclosure, the other additives include but are not limited to preservatives (e.g., NaN₃) and nuclease inhibitors. Preservatives are used for inhibition of the microbial contamination. Nuclease inhibitors are used for prevention of DNA degradation.

The working concentration of the Mg²⁺ may be 1.5 mM to 3 mM and may be adjusted based on experimental conditions.

To solve the above technical problems, the present disclosure further provides a biological material, where the biological material is any one selected from the group consisting of:
B1) a nucleic acid molecule encoding the above peptides;
B2) an expression cassette containing the nucleic acid molecule of B1);
B3) a recombinant vector containing the nucleic acid molecule of B1), or a recombinant vector containing the expression cassette of B2);
B4) a recombinant cell containing the nucleic acid molecule of B1), a recombinant cell containing the expression cassette of B2), or a recombinant cell containing the recombinant vector of B3);
B5) a recombinant tissue containing the nucleic acid molecule of B1), a recombinant tissue containing the expression cassette of B2), or a recombinant tissue containing the recombinant vector of B3);
B6) a recombinant organ containing the nucleic acid molecule of B1), a recombinant organ containing the expression cassette of B2), or a recombinant organ containing the recombinant vector of B3);
B7) a recombinant organism containing the nucleic acid molecule of B1), a recombinant organism containing the expression cassette of B2), or a recombinant organism containing the recombinant vector of B3); and
B8) a recombinant microorganism containing the nucleic acid molecule of B1), a recombinant microorganism containing the expression cassette of B2), or a recombinant microorganism containing the recombinant vector of B3).

In the nucleic acid molecule of B1), a person skilled in the art could readily employ known methods, such as directed evolution or site-directed mutagenesis, to mutate the nucleotide sequences encoding the polypeptide described above of the present disclosure. Those artificially modified nucleotide sequences exhibiting 80% or more than 80% identity to the nucleotide sequence of the polypeptide isolated in the present disclosure are all derived from and equivalent to the nucleotide sequences of the present disclosure provided they encode the polypeptide and retain the ability to inhibit cancer cell growth.

The aforementioned “80% or more than 80% identity” may refer to 81%, 82%, 83%, 84%, 85%, 86%, 87%, 88%, 89%, 90%, 91%, 92%, 93%, 94%, 95%, 96%, 97%, 98%, or 99% identity.

Herein, “identity” refers to the identity between amino acid sequences or nucleotide sequences. Sequence identity can be determined using homology search sites on the international internet, such as the BLAST page on the NCBI homepage website. For example, a determination method includes: in advanced BLAST2.1, by using BlastP as the program, setting the Expect value to 10, setting all Filters to OFF, using BLOSUM62 as the Matrix, setting Gap existence cost, Per residue gap cost, and Lambda ratio to 11, 1, and 0.85 (default values), respectively, and conducting pairwise alignment of amino acid sequences to calculate identity, thus obtaining the percentage identity value (%).

In the biological materials above, the nucleic acid molecule of B1) may be a coding gene for the protein. Specifically, the nucleic acid molecule of B1) may be a DNA molecule having the sequence of SEQ ID NO: 4 in a coding strand.

In the present disclosure, the vector is well-known to those skilled in the art and includes, but is not limited to: plasmids, phages (e.g., λ phage or M13 filamentous phage), cosmid (i.e., cos plasmid), Ti plasmids, or viral vectors.

In the biological materials above, the expression cassette of B2) refers to a DNA capable of expressing the gene in a host cell. This DNA may include not only a promoter to initiate transcription of the gene but also a terminator to halt transcription. Further, the expression cassette may further include an enhancer sequence.

Furthermore, the present disclosure further provides use of the biological material.

The use is any one selected from the group consisting of:
1) use of the biological material in preparation of a photosensitive DNA polymerase;
2) use of the biological material in preparation of a photosensitive DNA polymerase product; and
3) use of the biological material in isothermal nucleic acid amplification or in preparation of a product for the isothermal nucleic acid amplification.

The present disclosure further provides a kit, where the kit includes a protein prepared by any one of the preparation methods, a compound prepared by any one of the preparation methods, and/or the photosensitive oligonucleotide strand.

### Beneficial Effects

A photosensitive DNA polymerase, use method and use thereof are provided in the present disclosure, relate to the technical field of enzyme engineering, and address the technical challenges of providing a protein and converting the protein into the photosensitive DNA polymerase. The protein has the amino acid sequence set forth in SEQ ID NO: 3, where X is pAzF, an amino group of the pAzF is linked via a peptide bond to a carboxyl group of lysine at position 4 in SEQ ID NO: 3, and a carboxyl group of the pAzF is linked via a peptide bond to an amino group of histidine at position 5 in SEQ ID NO: 3. The photosensitive DNA polymerase is obtained by binding the protein to a photosensitive oligonucleotide strand set forth in SEQ ID NO: 5, which is capable of initiating an amplification reaction only upon irradiation at 365 nm, thereby enabling controlled activation of a DNA polymerase in vitro and rendering the DNA polymerase suitable for industrial production.

In the present disclosure, the key DNA polymerase in an isothermal nucleic acid amplification system is modified using non-natural amino acid method, enabling the enzymatic activity of the DNA polymerase to be controlled by light at specific wavelengths. The DNA polymerase is inactive prior to illumination and becomes activated upon illumination, thereby realizing photoinitiation of the isothermal nucleic acid amplification. An objective of the present disclosure is to provide a method for precisely controlling the initiation of isothermal nucleic acid amplification reactions. This approach addresses current limitations in isothermal amplification technologies and facilitates its in-depth application in precision fields such as nucleic acid detection and quantitative analysis.

### Beneficial Effects

A photosensitive DNA polymerase, use method and use thereof are provided, relate to the technical field of enzyme engineering, and address the technical challenges of providing a protein and converting the protein into the photosensitive DNA polymerase. The protein has the amino acid sequence set forth in SEQ ID NO: 3, where X is pAzF, an amino group of the pAzF is linked via a peptide bond to a carboxyl group of lysine at position 4 in SEQ ID NO: 3, and a carboxyl group of the pAzF is linked via a peptide bond to an amino group of histidine at position 5 in SEQ ID NO: 3. The photosensitive DNA polymerase is obtained by binding the protein to the photosensitive oligonucleotide strand set forth in SEQ ID NO: 5, exhibits the capability of initiating an amplification only upon irradiation at 365 nm, thereby enabling in vitro controlled initiation of the DNA polymerase and rendering the DNA polymerase suitable for industrial production.

In the present disclosure, the crucial DNA polymerase in an isothermal nucleic acid amplification system is modified using non-natural amino acid method, enabling its enzymatic activity to be controlled by specific wavelengths of light. The DNA polymerase is inactive prior to illumination and becomes activated upon exposure to the light, thereby achieving photoinitiated isothermal nucleic acid amplification. An objective of the present disclosure is to provide a method for precisely controlling the initiation of isothermal nucleic acid amplification reactions. This approach addresses current limitations in isothermal amplification technologies and facilitates its in-depth application in precision fields such as nucleic acid detection and quantitative analysis.

### BRIEF DESCRIPTION OF THE DRAWINGS

FIG. 1 shows schematic diagram of the experimental principle;

FIG. 2 shows structural formula of the non-natural amino acid pAzF;

FIG. 3 shows schematic diagram of the workflow for site-specific incorporation of an non-natural amino acid into a protein;

FIG. 4 is an image illustrating purified non-natural DNA polymerase;

FIG. 5 is a gel electrophoresis image illustrating the activity of the non-natural DNA polymerase;

FIG. 6 shows result of the mass spectrometry analysis of the natural DNA polymerase (measured molecular weight: 68,225.69 Da);

FIG. 7 shows result of the mass spectrometry analysis of the non-natural DNA polymerase (measured molecular weight: 68,402.82 Da);

FIG. 8 shows schematic diagram of the click chemistry reaction; and

FIG. 9 shows photoinitiated isothermal nucleic acid amplification.

### DETAILED DESCRIPTION OF THE EMBODIMENTS

The present disclosure will be described in further detail below with reference to specific examples. The examples given are only for the purpose of illustrating the present disclosure, and are not intended to limit the scope of the present disclosure. The examples provided below can serve as a guide for further improvement by those of ordinary skill in the art, and are not intended to limit the present disclosure in any way.

Unless otherwise specified, the experimental methods described in the following examples are all conventional methods, which are conducted in accordance with the techniques or conditions described in the literature in the art or in accordance with the product instructions. The materials, reagents, and the like used in the following examples are all commercially available, unless otherwise specified.

The data of the following examples are processed using SPSS 11.5 statistical software. Experimental results are expressed as mean±standard deviation. One-way ANOVA is employed for statistical analysis, with P<0.05 (*) indicating a significant difference, P<0.01 (**) indicating a highly significant difference, and P<0.001 (***) indicating an extremely significant difference.

The term “pAzF” refers to the compound with the structure shown in FIG. 2.

The term “azide group” denotes a functional group composed of three nitrogen atoms connected by double bonds (—N═N═N), with the chemical formula of —N3. The azide group exhibits a linear configuration in its chemical structure and is typically represented as N═N=N. In this application, the “N3” or “—N3” in the structural formula of the pAzF compound (FIG. 2) represents the azide group.

The term “aminoacyl-tRNA synthetase” refers to a class of enzymes that involves in the binding process of an amino acid to its corresponding tRNA molecule. The aminoacyl-tRNA synthetase-involving in the synthesis process occurs in two steps: first, the aminoacyl-tRNA synthetase recognizes the specific amino acid it catalyzes and another substrate ATP. Catalyzed by the aminoacyl-tRNA synthetase, carboxyl group of the amino acid bind to the phosphate group of ATP to form an ester bond, simultaneously releasing one molecule of pyrophosphate (PPi): Amino acid+ATP→Aminoacyl-AMP-enzyme intermediate+PPi, when the aminoacyl-AMP remains tightly bound to the enzyme molecule. Second, the aminoacyl-tRNA synthetase transfers the amino acid to the 3′-terminal ribose of tRNA via ester bond formation: Aminoacyl-AMP+tRNA→Aminoacyl-tRNA+AMP. Aminoacyl-tRNA synthetases differ in their tRNA recognition sites. Some aminoacyl-tRNA synthetases specifically form 2′-ester linkages, others form 3′-ester linkages, and some may form a mixture of both.

The terms “peptide bond linkage”, “peptide bond formation”, and “peptide bond” are synonymous and refer to the amide bond formed by the dehydration condensation between the α-carboxyl group of one amino acid and the α-amino group of another amino acid, represented as —CO—NH—.

The term “tRNA”, also known as “transfer RNA” “transfer ribonucleic acid”, is a type of RNA composed of 76-90 nucleotides. Its 3′-end can be catalyzed by the aminoacyl-tRNA synthetase to attach to a specific amino acid. During translation, tRNA recognizes codons on mRNA via its anticodon, delivering the corresponding amino acid to the ribosome for incorporation into the polypeptide chain being synthesized. While each tRNA molecule theoretically attaches specifically to one amino acid, the degeneracy of the genetic code enables more than one tRNA binding the same type of amino acid.

The term “amino acid arm” refers to a portion the tRNA molecule formed by base pairing between nucleotide sequences near its 3′-terminus and nucleotide sequences at 5′-terminus, which is primarily responsible for receiving an amino acid, and which can carry a specific amino acid to the ribosome to match the codon on the mRNA, ensuring their precise delivery to the polypeptide chain being synthesized.

The term “codon” refers to a group of three adjacent nucleotides in a messenger RNA (mRNA) molecule that specifies a certain amino acid during protein synthesis. The mRNAs in a cell determine the types and arrangements of amino acids in a protein. The sequence composed of the four nucleotides (bases) in mRNA dictates the sequence of 21 amino acids in proteins, and 3 bases in the mRNA molecule determine each amino acid. Examples include but are not limited to:

Codons UUU and UUC represent phenylalanine; UUA, UUG, CUU, CUC, CUA, and CUG represent leucine; UCU, UCC, UCA, UCG, AGU, and AGC represent serine; UAU, and UAC represent tyrosine; UGU, and UGC represent cysteine; UGG represents tryptophan; CCU, CCC, CCA, and CCG represent proline; CAU, and CAC represent histidine; CAA, and CAG represent glutamine; CGU, CGC, CGA, CGG, AGA, and AGG represent arginine; AUU, AUC, and AUA represent isoleucine; AUG represents methionine (initiation codon); ACU, ACC, ACA, and ACG represent threonine; AAU, and AAC represent asparagine; AAA, and AAG represent lysine; GUU, GUC, GUA, and GUG represent valine; GCU, GCC, GCA, and GCG represent alanine; GAU, and GAC represent aspartate; GAA, and GAG represent glutamate; GGU, GGC, GGA, and GGG represent glycine; UAA, UAG, and UGA: termination (termination codons).

The term “DNA polymerase” refers to a DNA-dependent DNA polymerase (DNA pol), an enzyme that catalyzes the polymerization of dNTPs into a daughter DNA strand, using a parental DNA molecule as a template.

The terms “photosensitive DNA polymerase” and “photoactivated DNA polymerase” are synonymous. They refer to a specialized DNA polymerase capable of initiating DNA replication under light irradiation. Such enzymes typically incorporate a photolabile group. Upon exposure to specific wavelengths of light, the photolabile group undergoes structural modification, thereby activating the enzyme's catalytic activity to drive DNA synthesis.

The term “gene” denotes a functional DNA segment involved in the generation of a polypeptide chain. It includes the regions flanking the coding sequence that involve in and regulate transcription/translation of the gene product (leader and trailer regions); as well as insertion sequence between individual coding regions (exons) (intron).

The terms “polypeptide”, “peptide”, and “protein” are used interchangeably herein to refer to a polymer of amino acid residues. This term applies to amino acid polymers in which one or more amino acid residues are artificial chemical analogs of the corresponding naturally occurring amino acids, as well as naturally occurring or non-naturally occurring amino acid polymers. As used herein, the term encompasses amino acid chains of any length, including full-length proteins (i.e., antigens) in which the amino acid residues are linked by covalent peptide bonds.

The term “amino acid” refers to both naturally occurring and synthetic amino acids, as well as amino acid analogs and mimetics that functionally resemble naturally occurring amino acids. Naturally occurring amino acids include those encoded by the genetic codes and post-translationally modified amino acids, such as hydroxyproline, γ-carboxyglutamate, and O-phosphoserine. Herein, amino acids may be designated using the standard three-letter abbreviations or single-letter codes recommended by the IUPAC-IUB Biochemical Nomenclature Commission. Similarly, nucleotides may be represented by their universally accepted single-letter codes.

The term “template” refers to any nucleic acid molecule usable for amplification as described in the present disclosure. Non-naturally occurring double-stranded RNA or DNA can be converted into double-stranded DNA to serve as a template. Any double-stranded DNA or articles containing many diverse double-stranded DNA molecules may be used as template DNA to amplify one or more loci of interest within the template DNA.

The term “primer” refers to an oligonucleotide used in amplification methods such as the polymerase chain reaction (PCR) to amplify nucleotide sequences based on a polynucleotide template corresponding to a specific genomic sequence. At least one PCR primer in a primer pair is sequence-specific to the target sequence for amplification.

The term “probe” denotes a molecule that binds to a specific sequence, subsequence, or region of another molecule. Unless otherwise specified, the term “probe” typically refers to a polynucleotide probe that binds to another polynucleotide (usually referred to as the “target polynucleotide”) via complementary base pairing. Probes may hybridize to target polynucleotides that lack complete sequence complementarity, which depends on the stringency of the hybridization conditions. Probes may be directly or indirectly labeled.

The term “amplification reaction” describes a process of copying the nucleic acid for one or more times. In specific embodiments, the amplification reaction includes, but is not limited to: PCR, self-sustained sequence replication, ligase chain reaction (LCR), and rapid amplification of cDNA ends (RACE), combined chain reaction (CCR), Q-β phage amplification, strand displacement amplification (SDA), and overlap extension polymerase chain reaction (OE-PCR). In some embodiments, single nucleic acid molecules are amplified, such as, through digital PCR (dPCR).

The term “amplification product” refers to nucleic acid product generated through nucleic acid amplification.

The term “kit” denotes any delivery system designed to deliver materials for a specific purpose.

The term “nucleic acid” describes a polymer, either single- or double-stranded, composed of at least two deoxyribonucleotides or ribonucleotides. Unless explicitly limited, the term encompasses nucleic acids including analogs of natural nucleotides, which retain similar binding properties and metabolic behavior with the reference nucleic acids thereof. Unless otherwise stated, a specific nucleic acid sequence implicitly includes its conservatively modified variants (e.g., with degenerate codon substitutions), alleles, orthologs, single nucleotide polymorphisms (SNPs), complementary sequences, and explicitly specified sequences. Specifically, those variants with degenerate codon substitutions may be achieved by generating the sequences in which the third position of one or more selected (or all) codons are replaced with mixed bases and/or deoxyinosine residues (Batzer et al., Nucleic Acids Res. 19:5081, 1991; Ohtsuka et al., J. Biol. Chem. 260:2605-2608, 1985; Cassol et al., 1992; Rossolini et al., Mol. Cell. Probes 8:91-98, 1994). The “nucleotide” consists of a sugar (deoxyribose for DNA or ribose for RNA), a nitrogenous base, and a phosphate group. Nucleotides are linked via phosphate bonds. The “nitrogenous base” includes purines and pyrimidines, encompassing natural compounds such as adenine, thymine, guanine, cytosine, uracil, inosine, and natural analogs, as well as purine and pyrimidine. The term “antigen” broadly refers to any immunogenic fragment or determinant to a selected target, including single or multiple epitopes, single or multiple domains, entire extracellular domains (ECDs), or full-length proteins. Antigens may be peptides, proteins, glycoproteins, polysaccharides, lipids, and fragments or combinations thereof. Non-limiting exemplary antigen includes tumor antigens or pathogen antigens. An antigen may also denote a molecule capable of eliciting an immune response. Any form of antigen, or cells/preparations containing the antigen, may be used to generate antibodies specific to the determinants of the antigen. Antigens may be isolated full-length proteins, cell surface proteins (e.g., obtained by immunizing with cells expressing at least a part of the antigen on its surface), soluble proteins (e.g., obtained by immunizing with only extracellular domain (ECD) of the proteins), or protein constructs (e.g., Fc-fusion antigens). The antigens may be produced in genetically modified cells. Any of the above antigens may be administered alone or together with one or more immunogenic adjuvants known in the art. DNA encoding the antigen may be genomic or non-genomic DNA (e.g., cDNA) and may encode at least a portion of the ECD that is sufficient to induce an immune response. Any expression vector may be used for transformation into the antigen-producing cells, and the expression vector include, but is not limited to, adenoviral vectors, lentiviral vectors, plasmids, and non-viral vectors such as cationic lipids.

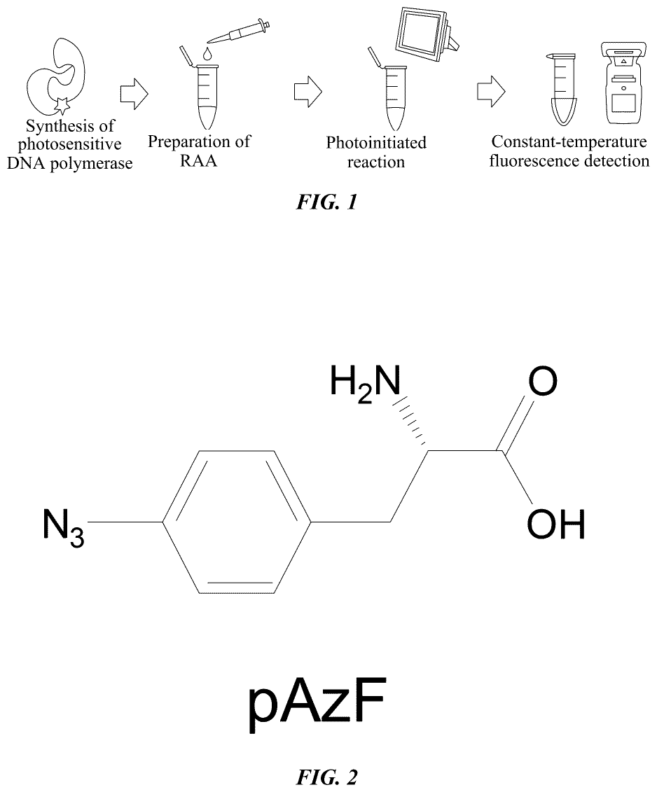

In isothermal nucleic acid amplification systems, DNA polymerase plays a central role in the nucleic acid amplification and exhibits activity at ambient temperatures. In the present disclosure, non-natural amino acids are introduced into the DNA polymerase, and oligonucleotide strands containing photolabile group are further conjugated, via azide groups of the non-natural amino acids. In the presence of the oligonucleotide strand, the DNA polymerase is inactive. Upon exposure to specific wavelengths of light, the photolabile group undergoes cleavage, releasing the oligonucleotide strand and restoring the activity, thereby generating the photosensitive DNA polymerase. The use of such an enzyme enables the large number of isothermal nucleic acid amplification systems have no reactivity and the non-specific amplification during preparation, and upon irradiation with specific wavelengths of light, the photosensitive DNA polymerase is activated post preparation, thus initiating the isothermal nucleic acid amplification, and realizing the initiation by light. This mechanism is as illustrated in FIG. 1.

In this application, the plasmids pZA16-pAzF and pEVOL-pAzF are identical. They are sourced from AddGene (Catalog No. 31186), where the plasmid is designated as pEVOL-pAzF, while it is referred to as pZA16-pAzF in the present application.

### EXAMPLES

The following examples are intended to exemplify the practice of embodiments of the disclosure but are by no means intended to limit the scope thereof.

### Example 1—Synthesis of Photosensitive DNA Polymerase

### (I) Synthesis of Photosensitive DNA Polymerase

### Expression and Purification of Non-Natural DNA Polymerase

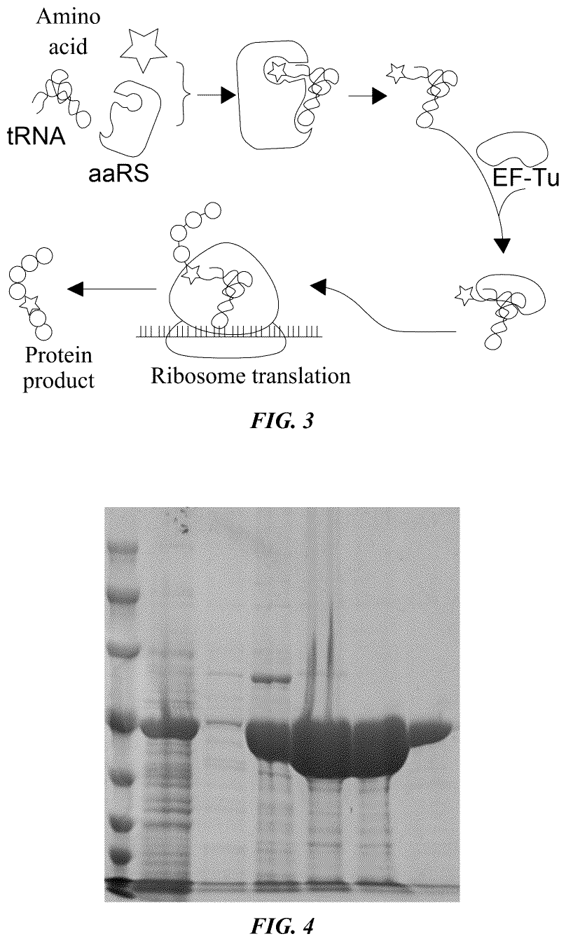

The non-natural amino acid used in the present disclosure was pAzF (structural formula shown in FIG. 2), which contained an azide group in its structure for subsequent conjugation with other molecules via click chemistry. To enable site-specific incorporation of the non-natural amino acid pAzF into the DNA polymerase at the same time, the protein-expressing cells were genetically modified to introduce the pZA16-pAzF plasmid. This plasmid expresses the aminoacyl-tRNA synthetase that is capable of recognizing the pAzF (pAzFRS) as well as a tRNATAG that is capable of recognizing the termination codon (TAG). The pAzFRS is capable of recognizing and aminoacylating the pAzF amino acid to attach it to the tRNATAG, thus forming a pAzF-tRNA-TAG complex. This complex is capable of entering into the ribosomes and involving in the protein translation, enabling site-specific insertion of the pAzF into the TAG codon to achieve non-natural modification of the DNA polymerase (synthetic schematic shown in FIG. 3).

Plasmid construction: the termination codon (TAG) was inserted into the coding sequence of the DNA polymerase protein (amino acid sequence of which was set forth in SEQ ID NO: 1) (the specific nucleotide sequence of the coding sequence, also known as the DNA polymerase protein gene, was set forth in SEQ ID NO: 2), i.e., between the 9th and 10th nucleotide residues of SEQ ID NO: 2. The nucleotide sequence of the modified DNA polymerase gene containing the inserted termination codon was set forth in SEQ ID NO: 4.

SEQ ID NO: 1 was as follows:
MRKHQHQHQHQHQSASVEDAIEKTIEIETSFDNVDFTSLKEAAIHFELDGGNYLRNNILK
FSLFTGEKHIVINADDINNYAELVSWLENPNTKKVVYDAKKTYVASHRLGIDIQNISFDIM
LASYIIDPSRTISDVQSVVSLYGQSFVKDDVSIYGKGKKFKVPEDDVLNPYVASITDAIYF
AKPNMDKQLEEYNQVELLADLELPLAKILSEMEEIGIFTDVHDLEEMEKEIQEKLDVLIR
NIHDAAGEDFNINSPKQLGVVLFETLQLPVIKKTKTGYSTAVDVLEQLQGEHPITDYILEY
RQLSKLQSTYVEGLQKVISDDQRIHTRFNQTLAQTGRLSSVDPNLQNIPVRLEEGRKIRK
AFKPTSKDSVILSADYSQIELRVLAHITQDESMKEAFINGDDIHTATAMKVFGVEADQVD
SSMRRQAKAINFGIVYGISDYGLSQSLGITRKKAKAFIDDYLASFPGVKQYMSDIVKDAK
ALGYVETLLHRRRYIPDITSRNFNLRGFAERTAMNTPIQGSAADIIKLAMVKFAQKMKET
TYQAKLLLQVHDELIFEVPKSEVDSFSEFVEEIMENALQLDVPLKVDSSYGATWYDAK.
SEQ ID NO: 2 was as follows:
ATGCGTAAACATCAGCATCAGCATCAGCATCAGCATCAGTCAGCAAGCGTTGAAGAT
GCAATAGAAAAGACAATTGAAATTGAAACGTCTTTTGATAATGTTGATTTTACTTCATT
GAAAGAAGCGGCCATCCATTTCGAATTAGACGGTGGTAACTATTTGCGAAATAATATT
TTAAAATTCTCTTTATTTACAGGTGAGAAACATATTGTAATTAATGCGGATGACATAAA
TAATTATGCCGAACTAGTTTCATGGTTAGAAAATCCGAATACGAAAAAAGTCGTATATG
ATGCTAAAAAAACATATGTAGCATCACATAGATTGGGAATTGATATTCAAAATATTTCT
TTCGATATTATGTTGGCCAGTTATATAATTGATCCATCTCGTACGATTAGTGATGTTCAA
TCAGTTGTTTCATTGTACGGTCAAAGTTTTGTGAAAGACGATGTGAGTATATATGGAA
AAGGTAAGAAATTTAAGGTACCTGAAGATGATGTTTTAAATCCATATGTTGCTTCTATT
ACTGATGCAATTTACTTTGCGAAACCAAATATGGATAAACAATTAGAAGAATACAATC
AGGTAGAACTCTTAGCTGATTTAGAGCTACCGCTAGCTAAGATTTTAAGTGAAATGGA
AGAAATTGGTATATTTACAGATGTTCATGATTTAGAAGAAATGGAAAAAGAAATTCAA
GAAAAATTAGACGTCTTGATTCGAAATATCCATGATGCAGCTGGTGAAGATTTTAATAT
AAATTCTCCTAAGCAATTAGGTGTTGTATTGTTTGAGACATTACAATTACCTGTTATTA
AAAAGACGAAAACAGGATATTCTACTGCTGTAGATGTCTTAGAGCAATTGCAAGGTG
AACATCCTATCACTGATTATATTTTAGAATATCGTCAATTATCTAAGTTACAGTCTACTT
ATGTTGAAGGATTGCAAAAGGTAATTAGTGATGATCAACGTATCCATACACGTTTTAA
CCAAACTTTAGCGCAAACTGGACGTTTATCAAGTGTAGATCCTAATTTACAAAATATTC
CGGTTAGACTTGAAGAAGGGCGTAAAATAAGAAAAGCCTTTAAACCAACTTCAAAA
GATAGCGTTATATTATCAGCAGATTATTCTCAAATTGAATTGCGTGTATTAGCACACATT
ACACAAGATGAGAGTATGAAAGAAGCATTTATCAACGGCGATGATATTCATACAGCAA
CTGCTATGAAAGTATTTGGTGTAGAAGCTGATCAAGTCGATAGTTCAATGCGTCGTCA
AGCAAAAGCGATTAACTTTGGAATTGTTTATGGGATAAGTGATTATGGTTTAAGTCAA
AGTTTAGGTATTACTCGTAAAAAAGCAAAAGCATTCATTGATGATTATTTAGCTAGTTT
CCCAGGTGTAAAACAATATATGTCTGATATTGTAAAAGATGCCAAAGCTTTAGGTTATG
TGGAAACATTGCTACATCGTCGACGCTATATTCCTGATATTACGAGTCGTAACTTTAAT
TTACGCGGCTTTGCTGAACGTACTGCTATGAATACGCCAATACAAGGCAGTGCTGCAG
ATATCATTAAACTGGCAATGGTTAAATTTGCACAAAAAATGAAAGAGACAACATATCA
AGCTAAACTATTATTACAAGTACACGATGAATTAATTTTTGAAGTACCTAAGTCAGAAG
TAGATTCATTTAGTGAATTTGTAGAAGAGATAATGGAAAATGCATTGCAATTAGATGTT
CCATTAAAAGTAGACTCAAGTTATGGTGCAACTTGGTATGATGCAAAATAA.
SEQ ID NO: 4 was as follows:
ATGCGTAAATAGCATCAGCATCAGCATCAGCATCAGCATCAGTCAGCAAGCGTTGAAG
ATGCAATAGAAAAGACAATTGAAATTGAAACGTCTTTTGATAATGTTGATTTTACTTCA
TTGAAAGAAGCGGCCATCCATTTCGAATTAGACGGTGGTAACTATTTGCGAAATAATA
TTTTAAAATTCTCTTTATTTACAGGTGAGAAACATATTGTAATTAATGCGGATGACATA
AATAATTATGCCGAACTAGTTTCATGGTTAGAAAATCCGAATACGAAAAAAGTCGTAT
ATGATGCTAAAAAAACATATGTAGCATCACATAGATTGGGAATTGATATTCAAAATATT
TCTTTCGATATTATGTTGGCCAGTTATATAATTGATCCATCTCGTACGATTAGTGATGTTC
AATCAGTTGTTTCATTGTACGGTCAAAGTTTTGTGAAAGACGATGTGAGTATATATGG
AAAAGGTAAGAAATTTAAGGTACCTGAAGATGATGTTTTAAATCCATATGTTGCTTCTA
TTACTGATGCAATTTACTTTGCGAAACCAAATATGGATAAACAATTAGAAGAATACAAT
CAGGTAGAACTCTTAGCTGATTTAGAGCTACCGCTAGCTAAGATTTTAAGTGAAATGG
AAGAAATTGGTATATTTACAGATGTTCATGATTTAGAAGAAATGGAAAAAGAAATTCA
AGAAAAATTAGACGTCTTGATTCGAAATATCCATGATGCAGCTGGTGAAGATTTTAAT
ATAAATTCTCCTAAGCAATTAGGTGTTGTATTGTTTGAGACATTACAATTACCTGTTATT
AAAAAGACGAAAACAGGATATTCTACTGCTGTAGATGTCTTAGAGCAATTGCAAGGT
GAACATCCTATCACTGATTATATTTTAGAATATCGTCAATTATCTAAGTTACAGTCTACT
TATGTTGAAGGATTGCAAAAGGTAATTAGTGATGATCAACGTATCCATACACGTTTTAA
CCAAACTTTAGCGCAAACTGGACGTTTATCAAGTGTAGATCCTAATTTACAAAATATTC
CGGTTAGACTTGAAGAAGGGCGTAAAATAAGAAAAGCCTTTAAACCAACTTCAAAA
GATAGCGTTATATTATCAGCAGATTATTCTCAAATTGAATTGCGTGTATTAGCACACATT
ACACAAGATGAGAGTATGAAAGAAGCATTTATCAACGGCGATGATATTCATACAGCAA
CTGCTATGAAAGTATTTGGTGTAGAAGCTGATCAAGTCGATAGTTCAATGCGTCGTCA
AGCAAAAGCGATTAACTTTGGAATTGTTTATGGGATAAGTGATTATGGTTTAAGTCAA
AGTTTAGGTATTACTCGTAAAAAAGCAAAAGCATTCATTGATGATTATTTAGCTAGTTT
CCCAGGTGTAAAACAATATATGTCTGATATTGTAAAAGATGCCAAAGCTTTAGGTTATG
TGGAAACATTGCTACATCGTCGACGCTATATTCCTGATATTACGAGTCGTAACTTTAAT
TTACGCGGCTTTGCTGAACGTACTGCTATGAATACGCCAATACAAGGCAGTGCTGCAG
ATATCATTAAACTGGCAATGGTTAAATTTGCACAAAAAATGAAAGAGACAACATATCA
AGCTAAACTATTATTACAAGTACACGATGAATTAATTTTTGAAGTACCTAAGTCAGAAG
TAGATTCATTTAGTGAATTTGTAGAAGAGATAATGGAAAATGCATTGCAATTAGATGTT
CCATTAAAAGTAGACTCAAGTTATGGTGCAACTTGGTATGATGCAAAATAA.

Specific experimental procedure was shown as follows:

Primer 1:
(SEQ ID NO: 10)
CATGCGTAAATAGCATCAGCATCAGCATCAGCATCAGCATCAGTCAG.
Primer 2:
(SEQ ID NO: 11)
GATGCTGATGCTATTTACGCATGGTCTGTTTCCTG

Template preparation: the DNA polymerase plasmid (pTrc99A-P) was subjected to DNA extraction using a plasmid extraction kit (TIANGEN DP103-02) to obtain the pTrc99A-P vector. The pTrc99A-P vector was digested with DpnI restriction enzyme (20 U/μL, New England Biolabs) at 37° C. overnight (12 hours) to generate the Trc99A-P vector digestion product, which served as the template for subsequent PCR.

PCR amplification: the template was amplified using the following PCR system and procedure to obtain PCR products.

A reaction system of the PCR was as follows:

TABLE 1

| | Reaction | | Final | |
|---|---|---|---|---|
| Component | system | | concentration | |
| TaKaRa-PrimeSTAR Max (TAKARA, | 25 | μl | 1X | |
| Catalog No: R045Q) | | | | |
| 10 μM Primer 1 | 5 | μl | 1 | μM |
| 10 μM Primer 2 | 5 | μl | 1 | μM |
| Template | 100 | ng | 100 | ng |
| ddH2O | to 50 | μl | | |

The PCR procedure was as follows:

TABLE 2

| Step | Temperature | Time |
|---|---|---|
| Denaturation | 98° C. | 30 seconds |
| 20 cycles | 98° C.; | 30 seconds; |
| | 55° C.; | 30 seconds; |
| | 72° C. | 7 min |
| Final extension | 72° C. | 15 minutes |
| Holding | 4° C. | |

### Transformation and Sequencing

A 5 μL aliquot of the PCR product was transformed into DH5a competent cells. Single colonies were picked after growth, and individual colonies were sent for sequencing. The correctly sequenced ligation product was preserved to obtain the plasmid pTrc99A-P4TAG of the non-natural DNA polymerase (also referred to as the pTrc99A-P4TAG recombinant vector).

The pTrc99A-P4TAG recombinant vector was constructed by inserting Fragment 3 (5′-TAG-3′) between Fragment 1 (5′-ATGCGTAAA-3′) and Fragment 2 (5′-CATCAGCATCAGCATCAGCATCAGCA-3′ (SEQ ID NO: 12)) of the pTrc99A vector (pTrc99A vector was purchased from HonorGene, Catalog No. HG-VYA0262), while retaining all other nucleotide sequences of the original pTrc99A vector. The resulting recombinant plasmid was designated as pTrc99A-P4TAG (also referred to as the pTrc99A-P4TAG recombinant vector).

### Plasmid Transformation and Strain Construction

The pTrc99A-P4TAG and pZA16-pAzF plasmids were sequentially transformed into the BL21 (DE3) strain in two steps, resulting in a dual-resistance strain capable of expressing the non-natural DNA polymerase. This strain was designated as BL21 (DE3)/(pTrc99A-P4TAG/pZA16-pAzF), where the BL21 (DE3)/(pTrc99A-P4TAG/pZA16-pAzF) referred to the BL21 (DE3) strain harboring both the pTrc99A-P4TAG and pZA16-pAzF plasmids.

### Example 2—Expression and Purification of DNA Polymerase

The structural formula of pAzF (CAS NO: 34670-43-4) was shown in FIG. 2.

The constructed strain (BL21 (DE3)/(pTrc99A-P4TAG/pZA16-pAzF)) was utilized for the expression and purification of the non-natural DNA polymerase as follows:
(1) A single colony of the (BL21 (DE3)/(pTrc99A-P4TAG/pZA16-pAzF)) was selected and cultured in LB medium containing Amp and Kan antibiotics with shaking until the OD 600 value reached 0.6. IPTG (final concentration 0.1 mM), pAzF (final concentration 0.1 mM), and L-arabinose (final concentration 10 mM) were then added, followed by continued incubation at 16° C. overnight (12 hours).
(2) The bacterial cells were harvested, and a portion was lysed and centrifuged. The supernatant and pellet were analyzed by SDS-PAGE, respectively to verify protein expression.
(3) Upon confirmation of protein expression, the remaining bacterial cells were lysed, and the lysate was purified using an AKTA nickel affinity column (HisTrap HP, cytiva, Catalog No. 17524802). The purified product was analyzed by SDS-PAGE to confirm protein expression, yielding the purified non-natural DNA polymerase.
(4) The purified non-natural DNA polymerase was dialyzed into the DNA polymerase dialysis buffer, resulting in a purified non-natural DNA polymerase solution with a protein concentration of 1 mg/L.

The amino acid sequence of the non-natural DNA polymerase was set forth in SEQ ID NO: 3. Where X was pAzF. Its structural formula was depicted in FIG. 2, and the amino group of the pAzF forming a peptide bond to the carboxyl group of the 4th lysine in SEQ ID NO: 3, and the carboxyl group of the pAzF forming a peptide bond to the amino group of the 5th histidine in SEQ ID NO: 3.

SEQ ID NO: 3 was as follows:
MRKXHQHQHQHQHQSASVEDAIEKTIEIETSFDNVDFTSLKEAAIHFELDGGNYLRNNIL
KFSLFTGEKHIVINADDINNYAELVSWLENPNTKKVVYDAKKTYVASHRLGIDIQNISFDI
MLASYIIDPSRTISDVQSVVSLYGQSFVKDDVSIYGKGKKFKVPEDDVLNPYVASITDAIY
FAKPNMDKQLEEYNQVELLADLELPLAKILSEMEEIGIFTDVHDLEEMEKEIQEKLDVLI
RNIHDAAGEDFNINSPKQLGVVLFETLQLPVIKKTKTGYSTAVDVLEQLQGEHPITDYILE
YRQLSKLQSTYVEGLQKVISDDQRIHTRFNQTLAQTGRLSSVDPNLQNIPVRLEEGRKIR
KAFKPTSKDSVILSADYSQIELRVLAHITQDESMKEAFINGDDIHTATAMKVFGVEADQV
DSSMRRQAKAINFGIVYGISDYGLSQSLGITRKKAKAFIDDYLASFPGVKQYMSDIVKDA
KALGYVETLLHRRRYIPDITSRNFNLRGFAERTAMNTPIQGSAADIIKLAMVKFAQKMKE
TTYQAKLLLQVHDELIFEVPKSEVDSFSEFVEEIMENALQLDVPLKVDSSYGATWYDAK.

The HQHQHQHQHQ (SEQ ID NO: 13) sequence at positions 5 to 14 of SEQ ID NO: 3 could bind to the nickel affinity column.

### Example 3—Activity Assay of the Non-Natural DNA Polymerase

Non-natural DNA polymerase: the purified non-natural DNA polymerase that was prepared as described above.

Natural DNA polymerase (also referred to as the natural DNA polymerase solution in the present disclosure): this was a component from the RAA reagent kit (Catalog No. MD001) provided by Qi's Academy, and the natural DNA polymerase solution contains the natural DNA polymerase with the amino acid sequence set forth in SEQ ID NO: 9.

SEQ ID NO: 9 was as follows:
MRKHQHQHQHQHQSASVEDAIEKTIEIETSFDNVDFTSLKEAAIHFELDGGNYLRNNILK
FSLFTGEKHIVINADDINNYAELVSWLENPNTKKVVYDAKKTYVASHRLGIDIQNISFDIM
LASYIIDPSRTISDVQSVVSLYGQSFVKDDVSIYGKGKKFKVPEDDVLNPYVASITDAIYF
AKPNMDKQLEEYNQVELLADLELPLAKILSEMEEIGIFTDVHDLEEMEKEIQEKLDVLIR
NIHDAAGEDFNINSPKQLGVVLFETLQLPVIKKTKTGYSTAVDVLEQLQGEHPITDYILEY
RQLSKLQSTYVEGLQKVISDDQRIHTRFNQTLAQTGRLSSVDPNLQNIPVRLEEGRKIRK
AFKPTSKDSVILSADYSQIELRVLAHITQDESMKEAFINGDDIHTATAMKVFGVEADQVD
SSMRRQAKAINFGIVYGISDYGLSQSLGITRKKAKAFIDDYLASFPGVKQYMSDIVKDAK
ALGYVETLLHRRRYIPDITSRNFNLRGFAERTAMNTPIQGSAADIIKLAMVKFAQKMKET
TYQAKLLLQVHDELIFEVPKSEVDSFSEFVEEIMENALQLDVPLKVDSSYGATWYDAK.

The HQHQHQHQHQ (SEQ ID NO: 14) sequence at positions 4 to 13 of SEQ ID NO: 9 could bind to the nickel affinity column.

The concentration of protein (natural DNA polymerase) in the natural DNA polymerase solution was 3.8 mg/L. The sole difference between the non-natural DNA polymerase (amino acid sequence shown in SEQ ID NO: 3) and the natural DNA polymerase (amino acid sequence shown in SEQ ID NO: 9) was the insertion of pAzF between the 3rd and 4th amino acids of the natural DNA polymerase. To assess whether the non-natural DNA polymerase could support the RAA reaction by replacing the natural DNA polymerase in the RAA system, the following experimental method was conducted:

The experiment was divided into two groups: the natural DNA polymerase RAA reaction group and the non-natural DNA polymerase RAA reaction group.

### Natural DNA Polymerase RAA Reaction Group:

Template amplification was conducted using the reaction Buffer listed in Table 4 and the reaction system outlined in Table 5.

### Non-Natural DNA Polymerase RAA Reaction Group:

The non-natural DNA polymerase RAA reaction group differed from the natural DNA polymerase RAA reaction group only in that the natural DNA polymerase was replaced with the non-natural DNA polymerase. All other procedures remained identical to those of the natural DNA polymerase RAA reaction group.

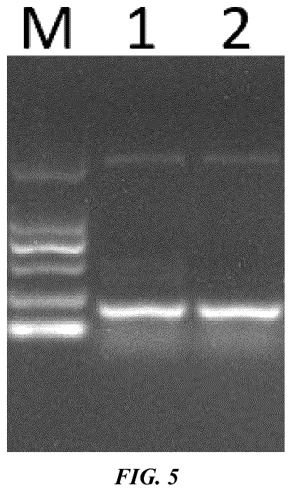

The results, as shown in FIG. 5 (in FIG. 5, Lane M represents the protein marker with bands corresponding to 100 bp, 250 bp, 500 bp, 750 bp, 1,000 bp, and 2,000 bp from top to bottom; Lane 1 corresponds to the natural DNA polymerase RAA reaction group; and Lane 2 corresponds to the non-natural DNA polymerase RAA reaction group), demonstrating that both the non-natural DNA polymerase RAA reaction group and the natural DNA polymerase RAA reaction group exhibited normal DNA polymerase activity.

### Example 4—Mass Spectrometric Analysis of the Non-Natural DNA Polymerase

To verify the insertion of the non-natural amino acid into the DNA polymerase, and the formation of the intended non-natural DNA polymerase, mass spectrometric analysis was conducted on the obtained non-natural DNA polymerase. The instruments and parameters used were listed in the table below.

TABLE 3

| Instruments | HPLC-Q-TOF-MS (Agilent, USA) | | | |
|---|---|---|---|---|
| Electrospray | 3.5 | KV | Fragmentor | 175 |
| voltage | | | | |
| Scanning range | Positive ion, primary 600-3200 (m/z) | | | |
| Data collection | profile | | | |
| mode | | | | |
| Gas Temp (° C.) | 325 | | Gas Flow (l/min) | 5 |
| Nebulizer(psig) | 35 | | Sheath Gas Temp | 325 |
| Chromatographic | 300SB-C8, 2.1 × 50 mm, | | Sheath Gas Flow | 7 |
| column | 3.5 micron | | | |
| Column oven | 50° | C. | Flow velocity | 0.2 mL/min |
| temperature | | | | |

| | | | |
|---|---|---|---|
| Mobile phase A | 0.1% FA in water | Mobile phase B | 0.1% FA in ACN |

| | | | | | | | | |
|---|---|---|---|---|---|---|---|---|
| HPLC | Time (min) | 0 | 2 | 8 | 12 | 16 | 17.1 | 20 |
| Gradient | A (%) | 98 | 98 | 50 | 2 | 2 | 98 | 98 |
| procedure of | B (%) | 2 | 2 | 50 | 98 | 98 | 2 | 2 |
| mobile phase | | | | | | | | |

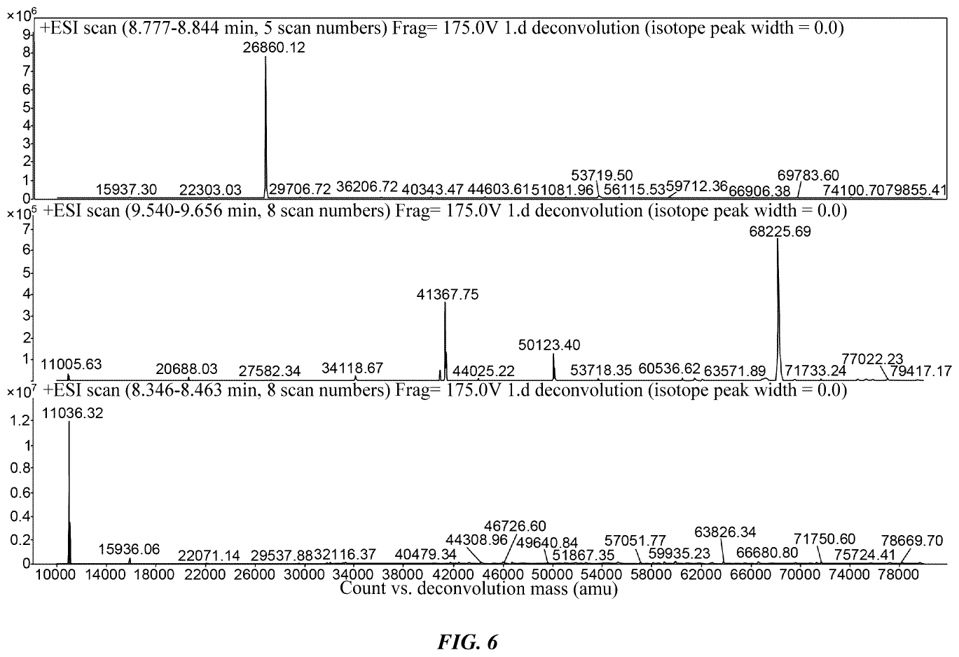

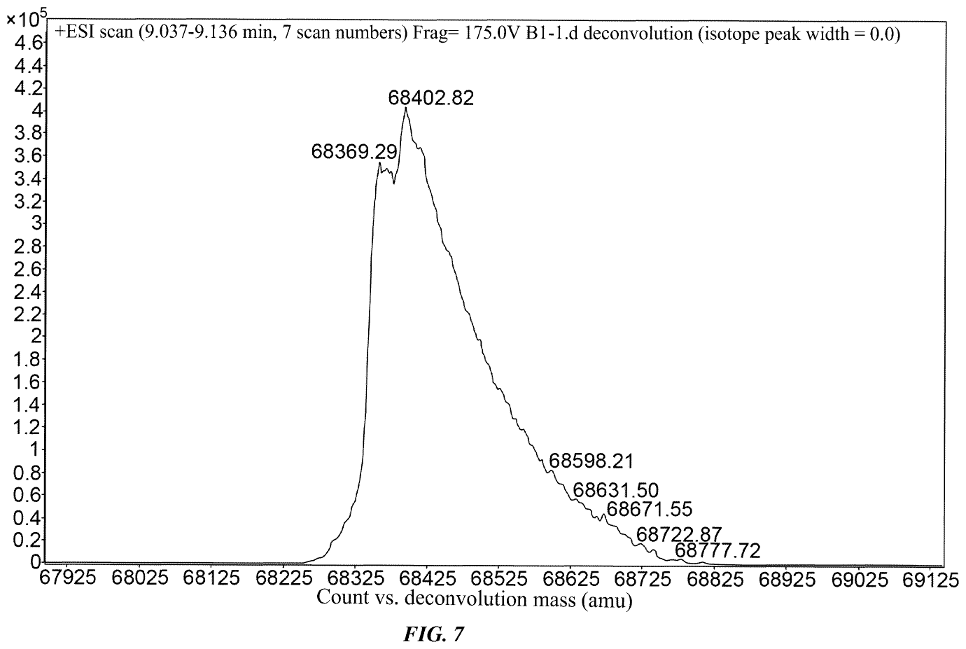

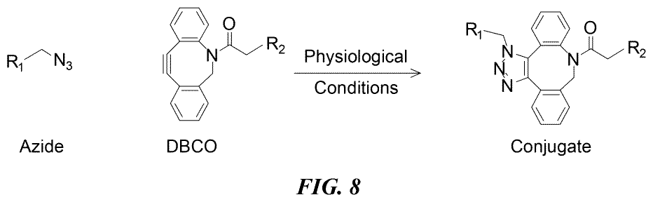

Samples (10 μL of the purified non-natural DNA polymerase solution and 10 μL of the purified natural DNA polymerase solution) were respectively loaded via an autosampler (7° C.), separated by HPLC, and subjected to mass spectrometric analysis. Profile spectra over the range of 600-3200 m/z were acquired. Deconvolution was conducted using the Deconvolute (MS): Protein software with the maximum entropy Deconvolute algorithm and a mass range of 10,000-80,000 Da. The measured molecular weight of the non-natural DNA polymerase was 68,402.82 Da (details are shown in FIG. 7, where the horizontal axis represents molecular weight and the vertical axis(s) represents signal intensity). Compared to the molecular weight of the wild-type DNA polymerase (natural DNA polymerase, 68,225.69 Da; details are shown in FIG. 6, with the same axis labels as FIG. 7), the non-natural DNA polymerase exhibited a molecular weight increase of 177.13 Da. Although this deviated from the theoretical difference of 188.2 Da by approximately 10 Da, the discrepancy fell within the acceptable margin of error for this instrument. Thus, it was concluded that the non-natural amino acid was successfully incorporated into the non-natural DNA polymerase, demonstrating the feasibility of this method.

### Example 5—Synthesis of Photosensitive Oligonucleotide Strand and Click Chemistry Conjugation

This application employed a strategy to “lock” the active site of the DNA polymerase by linking an oligonucleotide strand to the DNA polymerase, temporarily deactivating its enzymatic activity. A photosensitive oligonucleotide strand was synthesized and conjugated to the non-natural DNA polymerase in the RAA reaction via a click chemistry reaction to achieve locking and photoactivation of the enzymatic activity.

### Synthesis of the Photosensitive Oligonucleotide Strand

The photosensitive oligonucleotide strand was synthesized by GenScript Company and represented as:

TTCCTCTACCACCTACATCA [PC BMN] C-DBCO (SEQ ID NO:5), where the nucleotide sequence of SEQ ID NO: 5 was 5′-TTCCTCTACCACCTACATCA-3′

The specific structure of the photosensitive oligonucleotide strand was as follows:

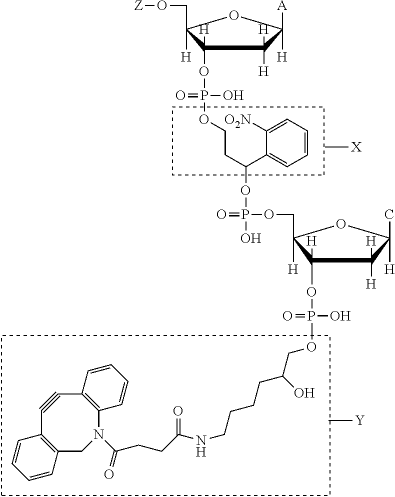

Where A represents adenine, which forms adenosine deoxyribonucleotide when links to deoxyribose; C represents cytosine, which forms cytidine deoxyribonucleotide when links to deoxyribose; X represents DBCO; Y represents [PC BMN]; Z is the oligonucleotide strand including the residues 1 to 19 of SEQ ID NO: 5; the phosphate group of the 3′-terminal cytosine deoxyribonucleotide of the Z forms the phosphodiester bond with the hydroxyl group on the C atom at position 5 of the adenine deoxyribonucleotide.

The photosensitive oligonucleotide strand was dissolved in water to prepare a photosensitive oligonucleotide solution with a concentration of 10 μM/L.

Here, PC BMN ([PC BMN]) served as the photolabile group, responsive to 365 nm wavelength light. Upon exposure to 365 nm light, this group underwent cleavage, causing the oligonucleotide strand to dissociate. DBCO acted as the click chemistry group, enabling conjugation via a click reaction with the azide residue of the pAzF in the non-natural DNA polymerase.

Conjugation of Non-Natural DNA Polymerase with the Photosensitive Oligonucleotide Strand
(1) The non-natural DNA polymerase and the photosensitive oligonucleotide strand were incubated at a 1:2 molar ratio in non-transparent tubes at 4° C. for 4 hours in a reaction buffer of 1×PBS. During this step, the non-natural DNA polymerase and the photosensitive oligonucleotide strand underwent conjugation via a click chemistry reaction.
(2) Following completion of step (1), unreacted DBCO-modified oligonucleotides (photosensitive oligonucleotide strands) were removed by ultrafiltration, yielding a solution of the non-natural DNA polymerase conjugated with the photosensitive oligonucleotide strand. The concentration of the conjugated non-natural DNA polymerase in the solution was 1 mg/mL.

The click chemistry reaction between the DBCO group in the photosensitive oligonucleotide strand and the azide group of the previously prepared non-natural DNA polymerase was illustrated in FIG. 8. The acetylene group of the DBCO in the photosensitive oligonucleotide strand undergoes an azide-acetylene Husigen cycloaddition reaction (i.e., Copper-Catalyzed Azide-Alkyne Cycloaddition) with the azide group of the pAzF in the above-prepared non-natural DNA polymerase.

Conjugation of Natural DNA Polymerase with the Photosensitive Oligonucleotide Strand

The conjugation of the natural DNA polymerase with the photosensitive oligonucleotide strand differed from that of the non-natural DNA polymerase only in that the purified non-natural DNA polymerase solution was replaced with the purified natural DNA polymerase solution. All other procedures remained identical to those described for the conjugation of the non-natural DNA polymerase with the photosensitive oligonucleotide strand. The resulting solution from the conjugation of the natural DNA polymerase with the photosensitive oligonucleotide strand had a natural DNA polymerase concentration of 1 mg/mL.

### Photoactivated Nucleic Acid Amplification in the RAA Reaction

### Preparation of the RAA System:

The RAA isothermal amplification system is currently a well-established nucleic acid amplification platform requiring protein components such as recombinase (X), recombinase accessory factor (Y), single-stranded DNA-binding protein (G), and DNA polymerase (P). This system has been commercialized by the collaborating company, Hangzhou Qi's Academy Future Biotechnology Co., Ltd., China, and is available as a high-quality reagent kit (catalog number MD001). In the present disclosure, the well-established and commercialized RAA isothermal amplification system was used and modified by replacing the native DNA polymerase (P) with the photoactivated non-natural DNA polymerase to achieve photoinitiated RAA reactions. The specific procedures were as follows:

### Photoinitiated RAA Reaction

(1) The photoactivated non-natural DNA polymerase was used to replace the general DNA polymerase provided in the RAA system to construct a photoinitiated isothermal amplification system. The template, primers, and system preparation were as follows:

### Template and Primers

Template: The amplification region of pET23a-sfGFPwt:

(SEQ ID NO: 6)
ACTAATGGTAAACTGACGCTGAAGTTCATCTGTACTACTGGTAAACTGCCGGT
ACCTTGGCCGACTCTGGTAACGACGCTGACTTATGGTGTTCAGTGCTTTGCTCGTTAT
CCGGACCATATGAAGCAGCATGACTTCTTCAAGTCCGCCATGCCGGAAGGCTATGTGC
AGGAACGCACGATTTCCTTTAAGGATGACGGCACGTACAAAACGCGTGCGGAAGTG
AAATTTGAAGGCGATACCCTGG.

The template was dissolved in water to prepare a template solution with a template concentration of 200 ng/μL.

### Primers:

RAA-1F:
(SEQ ID NO: 7)
ACTAATGGTAAACTGACGCTGAAGTTCATCTGTAC.
RAA-1R:
(SEQ ID NO: 8)
CCAGGGTATCGCCTTCAAATTTCACTTCCGCACGC.

### Preparation of the RAA System

The reaction buffer was prepared as specified in Table 4:

TABLE 4

| Buffer | Name | Formula/10 mL | Volume |
|---|---|---|---|
| RAA A Buffer | Tris- | 10 mL | 5.0 μL |
| | Ac | Tris 0.6 g, | |
| | | Potassium acetate 0.98 g, | |
| | | 2M DTT 250 μL, | |
| | | Adjusting pH to 8.3, | |
| | | Add ddH₂O to 10 mL, and store | |
| | | in a refrigerator at 4° C. | |
| RAA B Buffer | E-mix | 10 mL | 1.5 μL |
| | | ATP 0.3 g, | |
| | | PCr (phosphocreatine | |
| | | sodium tetrahydrate) 2.55 g, | |
| | | Add ddH₂O to 10 mL, and | |
| | | store in a refrigerator at | |
| | | −20° C. | |
| RAA C Buffer | CK | 10 mL | 2.5 μL |
| | | Creatine Kinase (CK) 20 mg, | |
| | | Add ddH₂O to 10 mL, | |
| | | and store in a refrigerator at | |
| | | −20° C. | |
| RAA D Buffer | dNTPs | 10 mM, store in a | 2.0 μL |
| | | refrigerator at −20° C. | |
| RAA E Buffer | PEG | 10 mL | 5.7 μL |
| | | PEG 35000 2 g, | |
| | | Add ddH₂O to 10 mL, and | |
| | | store in a refrigerator at | |
| | | 4° C. | |
| RAA F Buffer | D-T | 10 mL | 6.0 μL |
| | | D-T 5 g, | |
| | | Add ddH₂O to 10 mL, and | |
| | | store in a refrigerator at | |
| | | −20° C. | |

The reaction system was prepared as specified in Table 5:

TABLE 5

| Name/Buffer | Addition amount |
|---|---|
| RAA A Buffer | 5.0 μL |
| RAA B Buffer | 2.5 μL |
| RAA C Buffer | 2.5 μL |
| RAA D Buffer | 2.0 μL |
| RAA E Buffer | 5.7 μL |
| RAA F Buffer | 6.0 μL |
| X (recombinase; Hangzhou Qi's | 1 μL |
| Future Biotechnology Co., Ltd., kit | |
| catalog number MD001) | |
| Y (recombinase accessory factor; | 1 μL |
| Hangzhou Qi's Future Biotechnology | |
| Co., Ltd., kit catalog number MD001) | |
| G (single-stranded DNA-binding protein; | 1 μL |
| Hangzhou Qi's Future Biotechnology | |
| Co., Ltd., kit number MD001) | |
| P (non-natural DNA polymerase | 1 μL |
| solution conjugated with the | |
| photosensitive oligonucleotide strand) | |
| Primer 1 (RAA-1F; 10 μM); | 2 μL |
| Primer 1 (RAA-1R; 10 μM); | 2 μL |
| Template (template solution); | 1 μL |
| ddH₂O | add to 50 μL |
| Total volume | 50.0 μL |

The experiment was carried out in 5 groups: 0 s′ photoactivated native enzyme group, 300 s′ photoactivated native enzyme group, light-locked group, 180 s′ photoactivated group, and 300 s′ photoactivated group.

Procedure for the 0 s′ Photoactivated Native Enzyme Group:

Buffers were prepared according to Table 4, and the reaction system was prepared as specified in Table 5 (where P represents the natural DNA polymerase solution). After preparation, the mixture was thoroughly homogenized by inverting the tube 5-6 times with the cap closed, followed by low-speed centrifugation for 10 s. Without UV light irradiation, the reaction tube was placed into a 37° C. constant-temperature metal bath (or water bath/incubator) and incubated for 30 min. After the reaction, 10 μL of the reaction mixture was subjected to electrophoresis analysis. Prior to electrophoresis, the reaction mixture was purified using a mixed solution of phenol: chloroform: isoamyl alcohol (25:24:1), where the ratio of the reaction mixture to the mixed solution by volume was 1:1, followed by centrifugation at 12,000 rpm for 3-5 min. The supernatant was collected for electrophoresis to ensure optimal results. The electrophoresed gel was imaged using a gel imaging instrument.

Procedure for the 300 s′ Photoactivated Native Enzyme Group:

Buffers were prepared according to Table 4, and the reaction system was prepared as specified in Table 5 (where P represents the natural DNA polymerase solution). After preparation, the mixture was thoroughly homogenized by inverting the tube 5-6 times with the cap closed, followed by low-speed centrifugation for 10 s. The reaction tube was irradiated with a 35 W handheld UV lamp for 300 s. The reaction tube was then placed into a 37° C. constant-temperature metal bath (or water bath/incubator) and incubated for 30 min. After the reaction, 10 μL of the reaction mixture was subjected to electrophoresis analysis. Prior to electrophoresis, the reaction mixture was purified using a mixed solution of phenol: chloroform: isoamyl alcohol (25:24:1), where the ratio of the reaction mixture to the mixed solution by volume was 1:1, followed by centrifugation at 12,000 rpm for 3-5 min. The supernatant was collected for electrophoresis to ensure optimal results. The electrophoresed gel was imaged using a gel imaging instrument.

### Procedure for the Light-Locked Group:

Buffers were prepared according to Table 4, and the reaction system was prepared as specified in Table 5 (where P was replaced by the non-natural DNA polymerase solution conjugated with the photosensitive oligonucleotide strand). After preparation, the mixture was thoroughly homogenized by inverting the tube 5-6 times with the cap closed, followed by low-speed centrifugation for 10 s. Without UV light irradiation, the reaction tube was placed into a 37° C. constant-temperature metal bath (or water bath/incubator) and incubated for 30 min. After the reaction, 10 μL of the reaction mixture was subjected to electrophoresis analysis. Prior to electrophoresis, the reaction mixture was purified using a mixed solution of phenol: chloroform: isoamyl alcohol (25:24:1), where the ratio of the reaction mixture to the mixed solution by volume was 1:1, followed by centrifugation at 12,000 rpm for 3-5 min. The supernatant was collected for electrophoresis to ensure optimal results. The electrophoresed gel was imaged using a gel imaging instrument.

Procedure for the 180 s′ Photoactivated Group:

Buffers were prepared according to Table 4, and the reaction system was prepared as specified in Table 5 (where P was replaced by the non-natural DNA polymerase solution conjugated with the photosensitive oligonucleotide strand). After preparation, the mixture was thoroughly homogenized by inverting the tube 5-6 times with the cap closed, followed by low-speed centrifugation for 10 s. The reaction tube was irradiated with a 35 W handheld UV lamp for 180 s. The reaction tube was then placed into a 37° C. constant-temperature metal bath (or water bath/incubator) and incubated for 30 min. After the reaction, 5-10 μL (10 μL) of the reaction mixture was subjected to electrophoresis analysis. Prior to electrophoresis, the reaction mixture was purified using a mixed solution of phenol: chloroform: isoamyl alcohol (25:24:1), where the ratio of the reaction mixture to the mixed solution by volume was 1:1, followed by centrifugation at 12,000 rpm for 3-5 min. The supernatant was collected for electrophoresis to ensure optimal results. The electrophoresed gel was imaged using a gel imaging instrument.

Procedure for the 300 s′ Photoactivated Group:

Buffers were prepared according to Table 4, and the reaction system was prepared as specified in Table 5 (where P was replaced by the non-natural DNA polymerase solution conjugated with the photosensitive oligonucleotide strand). After preparation, the mixture was thoroughly homogenized by inverting the tube 5-6 times with the cap closed, followed by low-speed centrifugation for 10 s. The reaction tube was irradiated with a 35 W handheld UV lamp for 300 s. The reaction tube was then placed into a 37° C. constant-temperature metal bath (or water bath/incubator) and incubated for 30 min. After the reaction, 5-10 μL (10 μL) of the reaction mixture was subjected to electrophoresis analysis. Prior to electrophoresis, the reaction mixture was purified using a mixed solution of phenol: chloroform: isoamyl alcohol (25:24:1), where the ratio of the reaction mixture to the mixed solution by volume was 1:1, followed by centrifugation at 12,000 rpm for 3-5 min. The supernatant was collected for electrophoresis to ensure optimal results. The electrophoresed gel was imaged using a gel imaging instrument.

The electrophoretic images were subjected to gray value analysis using ImageJ, and the analyzed values were plotted using GraphPad Prism 9.5.

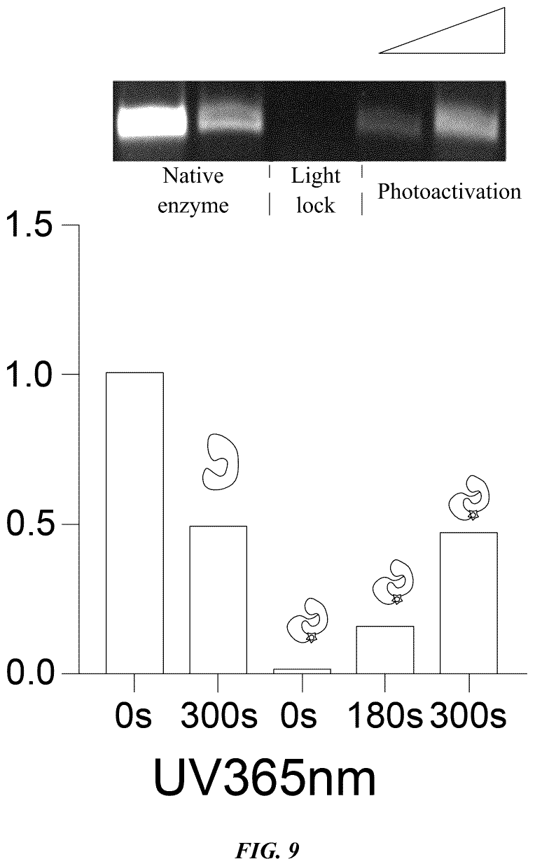

As shown in FIG. 9 (where in FIG. 9, the “native enzyme” represents from left to right lanes, to the 0 s′ photoactivated native enzyme group and the 300 s′ photoactivated native enzyme group, respectively; the “light lock” represents to the light-locked group, and the “photoactivation” represents, from left to right lanes, to the 180 s′ photoactivated group and the 300 s′ photoactivated group, respectively; the lanes of the gel image in the upper panel from left to right correspond to the 0 s′ photoactivated native enzyme group, 300 s′ photoactivated native enzyme group, light-locked group, 180 s′ photoactivated group, and 300 s′ photoactivated group; the bar graphs in the lower panel from left to right represent the 0 s′ photoactivated native enzyme group, 300 s′ photoactivated native enzyme group, light-locked group, 180 s′ photoactivated group, and 300 s′ photoactivated group, respectively). The results validate that the photoinitiated isothermal amplification system exhibited no activity in the absence of illumination; and upon irradiation with a 365 nm light, the successfully DNA amplied fragments were observed.

The present disclosure has been described in detail above. Without departing from the purpose and scope of the present disclosure and without unnecessary experimental conditions, the present disclosure can be implemented by those skilled in the art in a wide range under equivalent parameters, concentrations, and conditions. Although specific examples of the present disclosure have been given, it should be understood that the present disclosure can be further modified. In summary, according to the principle of the present disclosure, the present disclosure is intended to encompass any change to, use of, or modification to the present disclosure, including changes made using conventional techniques known in the art, which have departed from the scope disclosed in the present disclosure. Application of some basic features can be done in accordance with the scope of the following appended claims.

What is claimed is:

1. A protein, a salt thereof, or a derivative thereof, wherein the protein is any one selected from the group consisting of:
B1) a protein having the amino acid sequence set forth in SEQ ID NO: 3;
B2) a protein being obtained by substitution, and/or deletion, and/or addition of an amino acid residue in the protein of B1) having not less than 80% identity and retaining the same activity to the protein of B1); and
B3) a fusion protein being obtained by linking a protein tag to an N-terminus and/or a C-terminus of the protein of B1) or the protein of B2); wherein
X is pAzF, an amino group of the pAzF is linked via a peptide bond to a carboxyl group of lysine at position 4 in SEQ ID NO: 3, and a carboxyl group of the pAzF is linked via a peptide bond to an amino group of histidine at position 5 in SEQ ID NO: 3; and
the pAzF has the structure shown as follows:

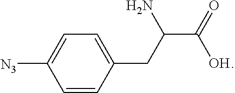 [SMILES: [N-]=[N+]=Nc1ccc(CC(N)C(=O)O)cc1]

2. A compound, wherein the compound has the structural formula as follows: 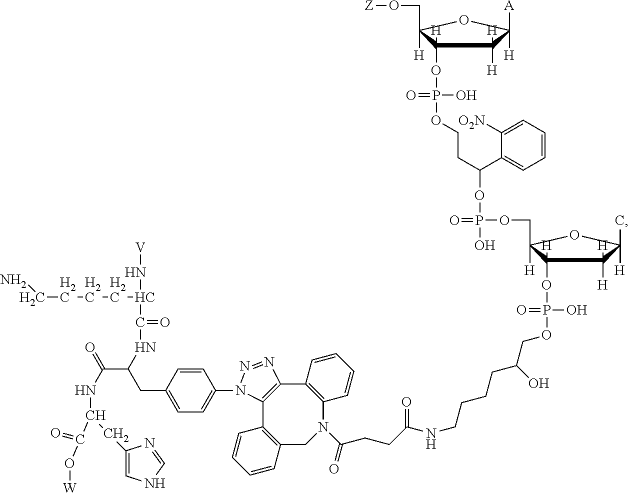
wherein
V is a polypeptide with an amino acid sequence of MR; W is a polypeptide comprising residues 5 to 598 of SEQ ID NO: 3; Z is an oligonucleotide strand comprising residues 1 to 19 of SEQ ID NO: 5; wherein
C represents cytosine; A represents adenine; a carboxyl group at the carboxyl terminus of the V forms a peptide bond with an amino group of a lysine residue; an amino group at the amino terminus of the W forms a peptide bond with a carboxyl group of a pAzF residue; and a phosphate group of a 3′-terminal cytosine deoxyribonucleotide of the Z forms a phosphodiester bond with an hydroxyl group on the C atom at position 5 of an adenine deoxyribonucleotide.

3. A method for preparing the protein according to claim 1, comprising:
transcribing and translating a nucleic acid having the nucleotide sequence set forth in SEQ ID NO: 4 to obtain the protein, wherein a pAzF-tRNA-TAG complex is involved in the translating process, and the pAzF-tRNA-TAG complex recognizes a TAG codon, and an amino arm of the pAzF-tRNA-TAG complex carries the pAzF according to claim 1.

4. The method according to claim 3, wherein the method comprises
transcribing and translating the nucleic acid having the nucleotide sequence set forth in SEQ ID NO: 4 in vitro to obtain the protein, wherein an aminoacyl-tRNA synthetase capable of recognizing the pAzF and a tRNA configured to recognize a termination codon is involved in the translating process.

5. The method according to claim 4, wherein the aminoacyl-tRNA synthetase capable of recognizing the pAzF and the tRNA configured to recognize the termination codon each are expressed via a pZA16-pAzF plasmid.

6. The method according to claim 3, wherein the transcribing and the translating each are conducted in a microorganism.

7. The method according to claim 6, wherein the method comprises
transcribing and translating the nucleic acid having the nucleotide sequence set forth in SEQ ID NO: 4 in vitro to obtain the protein, wherein an aminoacyl-tRNA synthetase capable of recognizing the pAzF and a tRNA configured to recognize a termination codon is involved in the translating process.

8. The method according to claim 7, wherein the aminoacyl-tRNA synthetase capable of recognizing the pAzF and the tRNA configured to recognize the termination codon each are expressed via a pZA16-pAzF plasmid.

9. A method for preparing the compound according to claim 2, comprising: subjecting an azide residue of the pAzF in a protein to copper-catalyzed azide-alkyne cycloaddition (CuAAC) with an alkyne group of dibenzocyclooctyne in a photosensitive oligonucleotide strand, thereby obtaining the compound according to claim 2; wherein
the protein is any one selected from the group consisting of:
B1) a protein having the amino acid sequence set forth in SEQ ID NO: 3;
B2) a protein being obtained by substitution, and/or deletion, and/or addition of an amino acid residue in the protein of B1) having not less than 80% identity and retaining the same activity to the protein of B1); and
B3) a fusion protein being obtained by linking a protein tag to an N-terminus and/or a C-terminus of the protein of B1) or the protein of B2); wherein
X is pAzF, an amino group of the pAzF is linked via a peptide bond to a carboxyl group of lysine at position 4 in SEQ ID NO: 3, and a carboxyl group of the pAzF is linked via a peptide bond to an amino group of histidine at position 5 in SEQ ID NO: 3; and
the pAzF has the structure shown as follows: 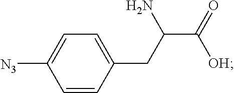 [SMILES: [N-]=[N+]=Nc1ccc(CC(N)C(=O)O)cc1]
and,
the photosensitive oligonucleotide strand has a structure as follows: 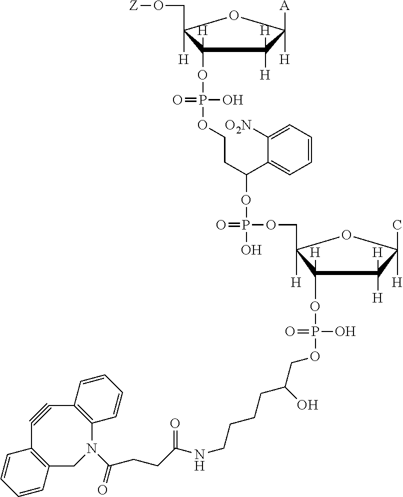
Z is the oligonucleotide strand comprising the residues 1 to 19 of SEQ ID NO: 5; C represents the cytosine; A represents the adenine; the phosphate group of the 3′-terminal cytosine deoxyribonucleotide of the Z forms the phosphodiester bond with the 5′-terminal hydroxyl group of the adenine deoxyribonucleotide.

10. A method for preparing a photosensitive DNA polymerase, comprising using the protein according to claim 1.

11. A method for performing isothermal nucleic acid amplification or preparing a product for the isothermal nucleic acid amplification, comprising using the protein according to claim 1.

12. A method for preparing a photosensitive DNA polymerase, comprising using the compound according to claim 2.

13. A method for performing isothermal nucleic acid amplification or preparing a product for the isothermal nucleic acid amplification, comprising using the compound according to claim 2.

14. A nucleic acid molecule encoding the protein according to claim 1.

15. An expression cassette comprising the nucleic acid molecule of according to claim 14.


SEQUENCE LISTING (14 sequences)

SEQ ID NO: 1 - protein, 601 aa, synthetic construct
  source 1..601: note = Amino acid sequence of the DNA polymerase protein
MRKHQHQHQHQHQSASVEDAIEKTIEIETSFDNVDFTSLKEAAIHFELDGGNYLRNNILKFSLFTGEKHIVINADDINNYAELVSWLENPNTKKVVYDAKKTYVASHRLGIDIQNISFDIMLASYIIDPSRTISDVQSVVSLYGQSFVKDDVSIYGKGKKFKVPEDDVLNPYVASITDAIYFAKPNMDKQLEEYNQVELLADLELPLAKILSEMEEIGIFTDVHDLEEMEKEIQEKLDVLIRNIHDAAGEDFNINSPKQLGVVLFETLQLPVIKKTKTGYSTAVDVLEQLQGEHPITDYILEYRQLSKLQSTYVEGLQKVISDDQRIHTRFNQTLAQTGRLSSVDPNLQNIPVRLEEGRKIRKAFKPTSKDSVILSADYSQIELRVLAHITQDESMKEAFINGDDIHTATAMKVFGVEADQVDSSMRRQAKAINFGIVYGISDYGLSQSLGITRKKAKAFIDDYLASFPGVKQYMSDIVKDAKALGYVETLLHRRRYIPDITSRNFNLRGFAERTAMNTPIQGSAADIIKLAMVKFAQKMKETTYQAKLLLQVHDELIFEVPKSEVDSFSEFVEEIMENALQLDVPLKVDSSYGATWYDAK

SEQ ID NO: 2 - DNA, 1806 bp, synthetic construct
  source 1..1806: mol_type = other DNA; note = Nucleotide sequence of the DNA polymerase protein gene
atgcgtaaacatcagcatcagcatcagcatcagcatcagtcagcaagcgttgaagatgcaatagaaaagacaattgaaattgaaacgtcttttgataatgttgattttacttcattgaaagaagcggccatccatttcgaattagacggtggtaactatttgcgaaataatattttaaaattctctttatttacaggtgagaaacatattgtaattaatgcggatgacataaataattatgccgaactagtttcatggttagaaaatccgaatacgaaaaaagtcgtatatgatgctaaaaaaacatatgtagcatcacatagattgggaattgatattcaaaatatttctttcgatattatgttggccagttatataattgatccatctcgtacgattagtgatgttcaatcagttgtttcattgtacggtcaaagttttgtgaaagacgatgtgagtatatatggaaaaggtaagaaatttaaggtacctgaagatgatgttttaaatccatatgttgcttctattactgatgcaatttactttgcgaaaccaaatatggataaacaattagaagaatacaatcaggtagaactcttagctgatttagagctaccgctagctaagattttaagtgaaatggaagaaattggtatatttacagatgttcatgatttagaagaaatggaaaaagaaattcaagaaaaattagacgtcttgattcgaaatatccatgatgcagctggtgaagattttaatataaattctcctaagcaattaggtgttgtattgtttgagacattacaattacctgttattaaaaagacgaaaacaggatattctactgctgtagatgtcttagagcaattgcaaggtgaacatcctatcactgattatattttagaatatcgtcaattatctaagttacagtctacttatgttgaaggattgcaaaaggtaattagtgatgatcaacgtatccatacacgttttaaccaaactttagcgcaaactggacgtttatcaagtgtagatcctaatttacaaaatattccggttagacttgaagaagggcgtaaaataagaaaagcctttaaaccaacttcaaaagatagcgttatattatcagcagattattctcaaattgaattgcgtgtattagcacacattacacaagatgagagtatgaaagaagcatttatcaacggcgatgatattcatacagcaactgctatgaaagtatttggtgtagaagctgatcaagtcgatagttcaatgcgtcgtcaagcaaaagcgattaactttggaattgtttatgggataagtgattatggtttaagtcaaagtttaggtattactcgtaaaaaagcaaaagcattcattgatgattatttagctagtttcccaggtgtaaaacaatatatgtctgatattgtaaaagatgccaaagctttaggttatgtggaaacattgctacatcgtcgacgctatattcctgatattacgagtcgtaactttaatttacgcggctttgctgaacgtactgctatgaatacgccaatacaaggcagtgctgcagatatcattaaactggcaatggttaaatttgcacaaaaaatgaaagagacaacatatcaagctaaactattattacaagtacacgatgaattaatttttgaagtacctaagtcagaagtagattcatttagtgaatttgtagaagagataatggaaaatgcattgcaattagatgttccattaaaagtagactcaagttatggtgcaacttggtatgatgcaaaataa

SEQ ID NO: 3 - protein, 602 aa, synthetic construct
  source 1..602: note = Amino acid sequence of the non-natural DNA polymerase
MRKXHQHQHQHQHQSASVEDAIEKTIEIETSFDNVDFTSLKEAAIHFELDGGNYLRNNILKFSLFTGEKHIVINADDINNYAELVSWLENPNTKKVVYDAKKTYVASHRLGIDIQNISFDIMLASYIIDPSRTISDVQSVVSLYGQSFVKDDVSIYGKGKKFKVPEDDVLNPYVASITDAIYFAKPNMDKQLEEYNQVELLADLELPLAKILSEMEEIGIFTDVHDLEEMEKEIQEKLDVLIRNIHDAAGEDFNINSPKQLGVVLFETLQLPVIKKTKTGYSTAVDVLEQLQGEHPITDYILEYRQLSKLQSTYVEGLQKVISDDQRIHTRFNQTLAQTGRLSSVDPNLQNIPVRLEEGRKIRKAFKPTSKDSVILSADYSQIELRVLAHITQDESMKEAFINGDDIHTATAMKVFGVEADQVDSSMRRQAKAINFGIVYGISDYGLSQSLGITRKKAKAFIDDYLASFPGVKQYMSDIVKDAKALGYVETLLHRRRYIPDITSRNFNLRGFAERTAMNTPIQGSAADIIKLAMVKFAQKMKETTYQAKLLLQVHDELIFEVPKSEVDSFSEFVEEIMENALQLDVPLKVDSSYGATWYDAK

SEQ ID NO: 4 - DNA, 1809 bp, synthetic construct
  source 1..1809: mol_type = other DNA; note = Nucleotide sequence of the modified DNA polymerase gene containing the inserted termination codon
atgcgtaaatagcatcagcatcagcatcagcatcagcatcagtcagcaagcgttgaagatgcaatagaaaagacaattgaaattgaaacgtcttttgataatgttgattttacttcattgaaagaagcggccatccatttcgaattagacggtggtaactatttgcgaaataatattttaaaattctctttatttacaggtgagaaacatattgtaattaatgcggatgacataaataattatgccgaactagtttcatggttagaaaatccgaatacgaaaaaagtcgtatatgatgctaaaaaaacatatgtagcatcacatagattgggaattgatattcaaaatatttctttcgatattatgttggccagttatataattgatccatctcgtacgattagtgatgttcaatcagttgtttcattgtacggtcaaagttttgtgaaagacgatgtgagtatatatggaaaaggtaagaaatttaaggtacctgaagatgatgttttaaatccatatgttgcttctattactgatgcaatttactttgcgaaaccaaatatggataaacaattagaagaatacaatcaggtagaactcttagctgatttagagctaccgctagctaagattttaagtgaaatggaagaaattggtatatttacagatgttcatgatttagaagaaatggaaaaagaaattcaagaaaaattagacgtcttgattcgaaatatccatgatgcagctggtgaagattttaatataaattctcctaagcaattaggtgttgtattgtttgagacattacaattacctgttattaaaaagacgaaaacaggatattctactgctgtagatgtcttagagcaattgcaaggtgaacatcctatcactgattatattttagaatatcgtcaattatctaagttacagtctacttatgttgaaggattgcaaaaggtaattagtgatgatcaacgtatccatacacgttttaaccaaactttagcgcaaactggacgtttatcaagtgtagatcctaatttacaaaatattccggttagacttgaagaagggcgtaaaataagaaaagcctttaaaccaacttcaaaagatagcgttatattatcagcagattattctcaaattgaattgcgtgtattagcacacattacacaagatgagagtatgaaagaagcatttatcaacggcgatgatattcatacagcaactgctatgaaagtatttggtgtagaagctgatcaagtcgatagttcaatgcgtcgtcaagcaaaagcgattaactttggaattgtttatgggataagtgattatggtttaagtcaaagtttaggtattactcgtaaaaaagcaaaagcattcattgatgattatttagctagtttcccaggtgtaaaacaatatatgtctgatattgtaaaagatgccaaagctttaggttatgtggaaacattgctacatcgtcgacgctatattcctgatattacgagtcgtaactttaatttacgcggctttgctgaacgtactgctatgaatacgccaatacaaggcagtgctgcagatatcattaaactggcaatggttaaatttgcacaaaaaatgaaagagacaacatatcaagctaaactattattacaagtacacgatgaattaatttttgaagtacctaagtcagaagtagattcatttagtgaatttgtagaagagataatggaaaatgcattgcaattagatgttccattaaaagtagactcaagttatggtgcaacttggtatgatgcaaaataa

SEQ ID NO: 5 - DNA, 20 bp, synthetic construct
  source 1..20: mol_type = other DNA; note = Nucleotide sequence of the photosensitive oligonucleotide strand
ttcctctaccacctacatca

SEQ ID NO: 6 - DNA, 247 bp, synthetic construct
  source 1..247: mol_type = other DNA; note = The amplification region of pET23a-sfGFPwt
actaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctgg

SEQ ID NO: 7 - DNA, 35 bp, synthetic construct
  source 1..35: mol_type = other DNA; note = Primer RAA-1F
actaatggtaaactgacgctgaagttcatctgtac

SEQ ID NO: 8 - DNA, 35 bp, synthetic construct
  source 1..35: mol_type = other DNA; note = Primer RAA-1R
ccagggtatcgccttcaaatttcacttccgcacgc

SEQ ID NO: 9 - protein, 601 aa, synthetic construct; identical to SEQ ID NO: 1
  source 1..601: note = Amino acid sequence of natural DNA polymerase

SEQ ID NO: 10 - DNA, 47 bp, synthetic construct
  source 1..47: mol_type = other DNA; note = Primer 1
catgcgtaaatagcatcagcatcagcatcagcatcagcatcagtcag

SEQ ID NO: 11 - DNA, 35 bp, synthetic construct
  source 1..35: mol_type = other DNA; note = Primer 2
gatgctgatgctatttacgcatggtctgtttcctg

SEQ ID NO: 12 - DNA, 26 bp, synthetic construct
  source 1..26: mol_type = other DNA; note = Fragment 2
catcagcatcagcatcagcatcagca

SEQ ID NO: 13 - protein, 10 aa, synthetic construct
  source 1..10: note = Positions 5 to 14 of SEQ ID NO: 3
HQHQHQHQHQ

SEQ ID NO: 14 - protein, 10 aa, synthetic construct
  source 1..10: note = Positions 4 to 13 of SEQ ID NO: 9
HQHQHQHQHQ